(19) United States
(12) Patent Application Publication
(10) Pub. No.: US 2026/0002939 A1
(43) Pub. Date: Jan. 1, 2026

(54) METHODS AND SYSTEMS FOR SENSITIVE AND MULTIPLEXED ANALYSIS OF BIOLOGICAL SAMPLES USING CLEAVABLE FLUORPHORES AND CLICK CHEMISTRY

(71) Applicant: ARIZONA BOARD OF REGENTS ON BEHALF OF ARIZONA STATE UNIVERSITY, Scottsdale, AZ (US)

(72) Inventors: Jia GUO, Tempe, AZ (US); Joshua LABAER, Chandler, AZ (US); Thai PHAM, Scottsdale, AZ (US); Yi CHEN, Scottsdale, AZ (US)

(21) Appl. No.: 19/106,987

(22) PCT Filed: May 1, 2024
(86) PCT No.: PCT/US2024/027185

§ 371 Date: Feb. 26, 2025

Related U.S. Application Data
(60) Provisional application No. 63/509,228, filed on Jun. 20, 2023.

(51) Int. Cl. G01N 33/68; C09B 23/08

(52) U.S. Cl. CPC G01N 33/6803; C09B 23/083

(57) ABSTRACT

Disclosed herein are compositions, systems, platforms, and kits comprising a tyramide-TCO and a tetrazine molecule linked to a detectable marker. Also disclosed are methods of using the compositions, systems, platforms, and kits to detect one or more target molecules, such as proteins or nucleic acids, in a sample.

### CROSS-REFERENCE TO RELATED APPLICATIONS

This application claims priority to U.S. Provisional Patent Application No. 63/509,228, filed Jun. 20, 2023, the content of which is hereby incorporated by reference in its entirety.

### STATEMENT REGARDING FEDERALLY SPONSORED RESEARCH

This invention was made with government support under R01 GM127633 awarded by the National Institutes of Health. The government has certain rights in the invention.

### BACKGROUND OF THE INVENTION

Single-cell spatial proteomic analysis holds great promise to advance our understanding of the composition, organization, interaction and function of the various cell types in complex biological systems. However, the current multiplexed protein imaging technologies suffer from their low detection sensitivity, multiplexing capacity or technically demanding. These deficiencies in the art drive demand for a highly sensitive and multiplexed in situ protein profiling method using off-the-shelf antibodies.

### BRIEF SUMMARY OF THE INVENTION

Disclosed herein is a system, kit, platform, or composition including a first component comprising a tyramide conjugated to a trans-cylooctene; and a second component comprising a detectable marker, a cleavable linker, and a tetrazine residue.

Disclosed herein is a compound having Formula (I):

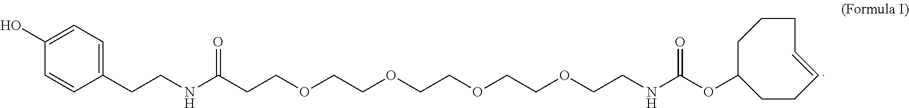 [SMILES: O=C(CCOCCOCCOCCOCCNC(=O)OC1CC/C=C/CCC1)NCCc1ccc(O)cc1]

Disclosed herein is a compound having Formula (II):

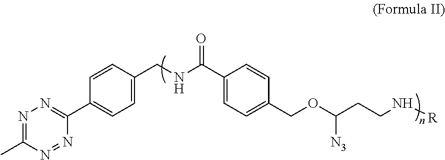

wherein R comprises a detectable marker; and
wherein n is 1 to 3.

Further disclosed herein is a compound having Formula (III):

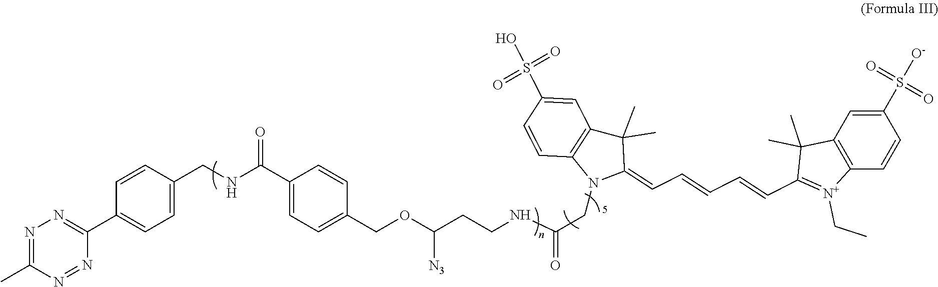

wherein n is 1 to 3.

Further disclosed herein is a method of multiplex in situ analysis of biomolecules in a tissue sample, the method including: (a) contacting a tissue sample with a first plurality of horseradish peroxidase (HRP)-conjugated targeting agents that are configured to specifically bind to or hybridize to a first target biomolecule in the tissue sample under conditions that promote binding or hybridization of the targeting agents to the target biomolecule; (b) contacting the tissue sample with the compound of Formula I under conditions that promote conjugation of the compound to the target biomolecule; (c) contacting the tissue sample with the compound of any one of claims 3-7 under conditions that promote conjugation of the compound of any one of claims 3-7 to the compound of Formula I; (d) imaging the tissue sample thereby detecting the detectable marker; (e) contacting the tissue sample with a composition comprising 1,3,5-Triaza-7-phosphaadamantane (PTA) and tris(2-carboxyethyl) phosphine (TCEP); (f) repeating steps (a)-(e); wherein a second plurality of HRP-conjugated targeting agents is used to bind to or hybridize to a second target biomolecule, wherein the first and the second target biomolecules are different.

Also disclosed herein is a kit comprising: (a) a composition comprising the compound of Formula I and Formula II; (b) a composition comprising 1,3,5-Triaza-7-phosphaadamantane (PTA); and (c) a composition comprising tris(2-carboxyethyl) phosphine (TCEP).

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1: Ultrasensitive and multiplexed protein imaging with cleavable fluorescent tetrazine (CFTz). In each cycle, multiple protein targets are first recognized by different primary antibodies. Subsequently, the first target is stained by HRP-conjugated antibodies and TCO-tyramide. The Cleavable fluorescent tetrazine (CFTz) are then covalently coupled to the TCO-tyramide via click chemistry reaction to stain the target protein. After imaging, the fluorophores are chemically cleaved and HRP is simultaneously deactivated. The processes of protein staining, fluorescence imaging, fluorophore cleavage and HRP deactivation are repeated until every target in the first cycle is stained. Finally, all the antibodies are stripped to initiate the next cycle. Through reiterative analysis cycles, a large number of distinct proteins can be quantitatively profiled in single cells in situ.

FIG. 2: Molecular structures of Cy5-linker-tetrazine, Cy5-2linkers-tetrazine, Cy5-3linkers-Tetrazine (fluorophore compartments) and TCO-Tyramide (Tyramide Compartments).

FIG. 3: Panel (A) shows the signal intensities obtained by staining protein CD45 with 1-linker, 2-linker, and 3-linker CFTz in human tonsil FFPE tissues for 10, 30 and 60 min, respectively. Panel (B) shows protein CD45 in human tonsil FFPE tissues is stained by conventional tyramide signal amplification (TSA), 1-linker, 2-linker, or 3-linker CFTz. Scale bars, 10 μm. Panel (C) shows comparison of the staining intensities generated by TSA, 1-linker, 2-linker, and 3-linker CFTz.

FIG. 4: Panel (A) shows protein CD45 is stained with 1-linker, 2-linker, and 3-linker CFTz in human tonsil FFPE tissues (left). Subsequently, the fluorescence signals are removed by mild reducing reagents (middle). Afterwards, the same tissues are blocked with free tetrazine and restained with CFTz (right). Scale bars, 10 μm. Panel (B) shows fluorescence intensity profiles corresponding to the indicated arrow positions in panel (A). Panel (C) shows a comparison of the cleavage efficiencies for 1 linker, 2 linker, and 3 linker CFTz.

FIG. 5: Panel (A) shows the 28 different proteins are stained with CFTet on the same FFPE Tissue. Scale bar, 500 μm. Panel (B) shows a zoomed view in the boxed area. Scale bar, 100 μm.

FIG. 6: Panel (A) shows based on their different single cell protein expression profiles, Panel (B) shows ˜820,000 individual cells in the human tonsil tissue are partitioned into 14 cell clusters. Panel (C) shows anatomical locations of each cell from the 14 clusters. Scale bar, 500 μm.

FIG. 7: Shows averaged cell numbers of each cell cluster in different cell neighborhoods.

FIG. 8: Panel (A) shows based on their neighbor cells from different clusters, panel (B) shows the individual cells in cluster 13 are further partitioned into 2 subclusters. Panel (C) shows anatomical locations of each cell from the three subclusters.

FIG. 9: shows structures of exemplary, non-limiting fluorescent detectable markers and linkers.

FIG. 10: shows 500 MHz ¹H NMR spectra of Tetrazine-N₃-Cy5 in CD₃OD.

FIG. 11: shows 500 MHz ¹H NMR spectra of Tetrazine-N₃—N₃-Cy5 in CD₃OD.

FIG. 12: shows 500 MHz ¹H NMR spectra of Tetrazine-N₃—N₃—N₃-Cy5 in CD₃OD.

FIG. 13: shows single cell protein expression distribution in Optsne plots.

FIG. 14: shows anatomical locations of the individual cells from different cell clusters.

FIG. 15: shows Scheme 2.

FIG. 16: shows Scheme 3.

FIG. 17: shows Scheme 4.

FIG. 18: shows Scheme 5.

### DETAILED DESCRIPTION

The system, kit, platform, or composition and compounds provided herein are based at least in part on the inventors' development of a highly sensitive and multiplexed in situ protein analysis approach that uses a first component, including Formula I, and a second component including Formulas II and III, which may herein also be referred to as “Cleavable Fluorescent Tetrazine” (CFTet) or cleavable detectably-labeled tetrazine. The system, kit, platform, or composition and compounds provided herein have the potential to quantify numerous different proteins and/or nucleic acids in individual cells of intact tissues at the optical resolution. As described herein, this development provides for in situ analysis of proteins, nucleic acids, and other biomolecules in intact tissues with single-molecule sensitivity.

Accordingly, in a first aspect, provided herein is a tyramide conjugated to a trans-cylooctene, herein referred to as TCO-tyramide. The TCO-tyramide is configured to permit recognition of tyramide by horseradish peroxidase (HRP) and to avoid compromised diffusion of a short-lived tyramide radical. In some embodiments, the TCO-tyramide comprises the structure of Formula I:

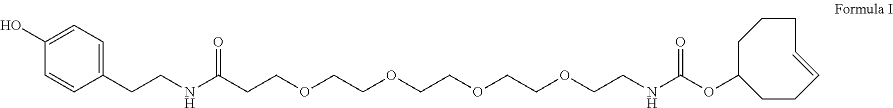 [SMILES: O=C(CCOCCOCCOCCOCCNC(=O)OC1CC/C=C/CCC1)NCCc1ccc(O)cc1]

The synthesis and characterization of TCO-tyramide is described in Example 1.

Also provided herein is a cleavable detectably-labeled tetrazine. In certain embodiments, a detectable label such as a fluorophore is tethered to tetrazine via a cleavable linker. In some embodiments, the cleavable linker is a chemically cleavable linker. As described herein, to allow for signal removal (e.g., fluorescent signal) after protein staining the cleavable detectably labeled tetrazine, in some embodiments, comprises a fluorophore tethered to tetrazine through a chemically cleavable linker. An aspect of the technology of this disclosure is efficient cleavage of the detectable label in a cellular environment while maintaining protein antigenicity. In some embodiments, the cleavable linker comprises the structure of Formula IV, wherein R includes a detectable marker, T is tetrazine, and n may be 1 to 3:

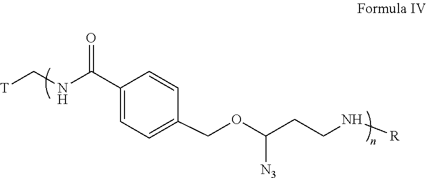

Other cleavable linkers appropriate for use in a CFTet of this disclosure include, without limitation, structures cleaved by enzymes, nucleophiles, electrophiles, reducing reagents, oxidizing reagents, photo-irradiation, metal catalysis, and the like. Further examples of suitable linkers and cleavage mechanisms are described by Milton et al. (U.S. Pat. No. 7,414,116) and by Leriche et al. (Bioorg. Med. Chem., 2012, 20:571-582), which are incorporated herein by reference in their entirety. The linker may be cleavable using a variety of approaches including the addition of a chemical agent, irradiation with one or more wavelengths of light, enzymatic reaction and the like.

In some embodiments, a cleavable detectably-labeled tetrazine is tetrazine-(N₃)ₙ—R, having the following chemical structure, Formula (II):

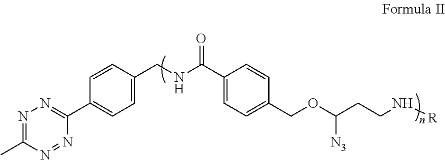

R is a detectable marker; and n is 1 to 3.

In some embodiments, the cleavable detectably-labeled tetrazine is tetrazine-(N₃)ₙ-Cy5 of Formula III:

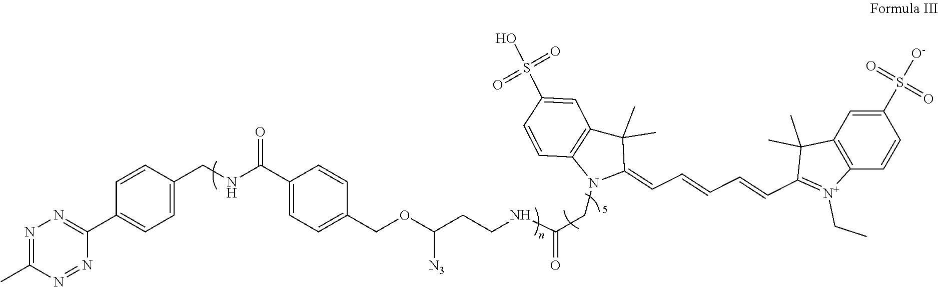

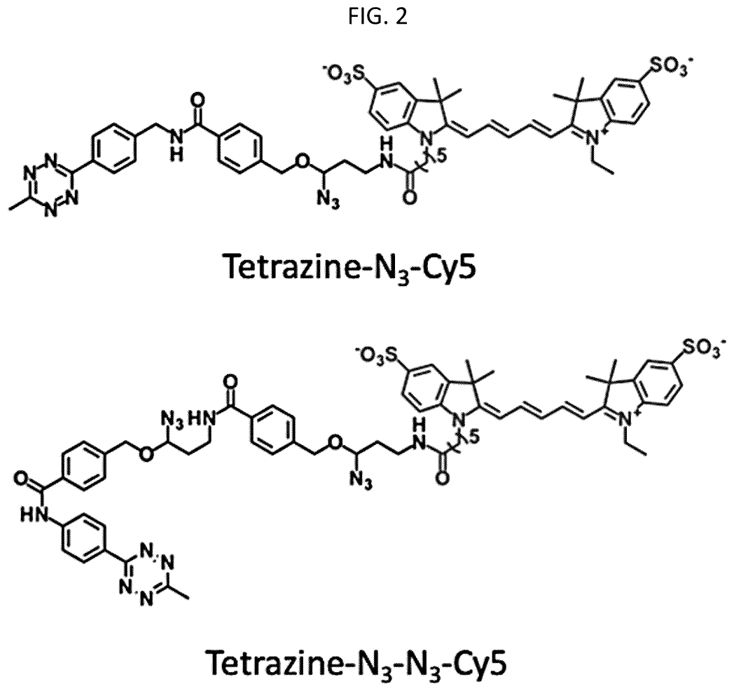

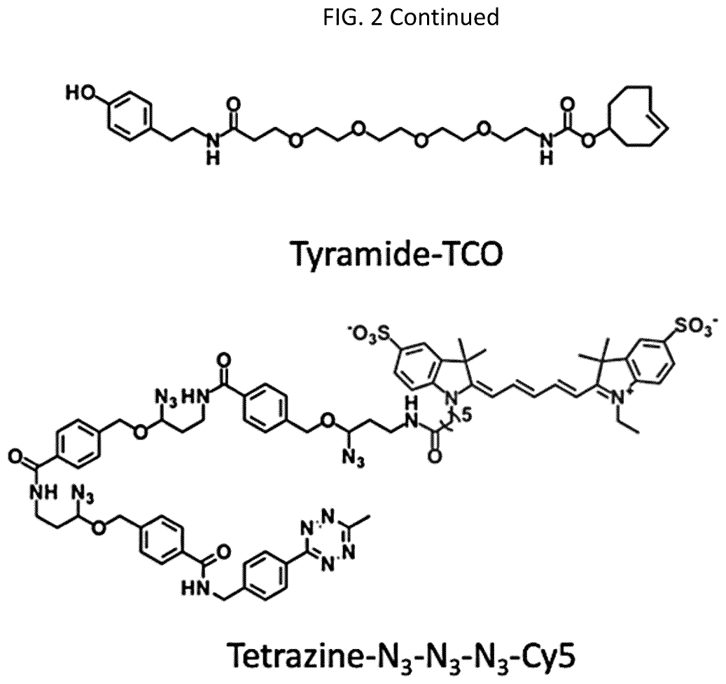

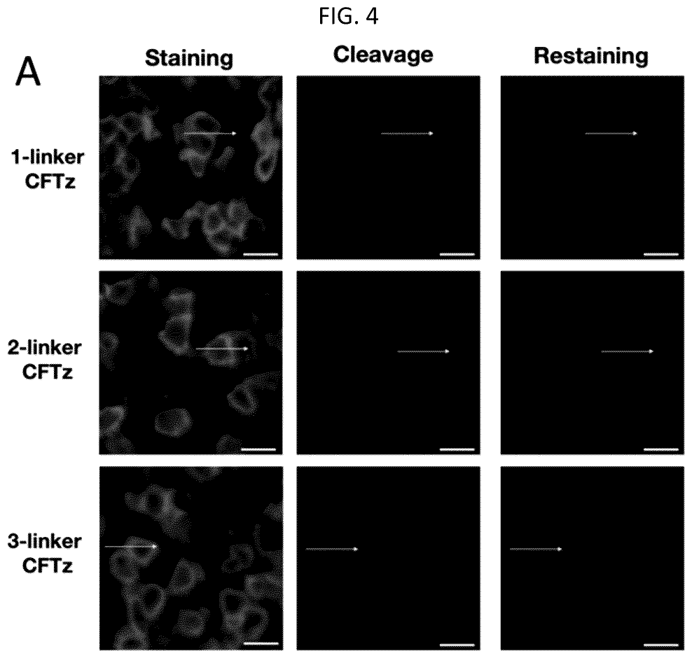

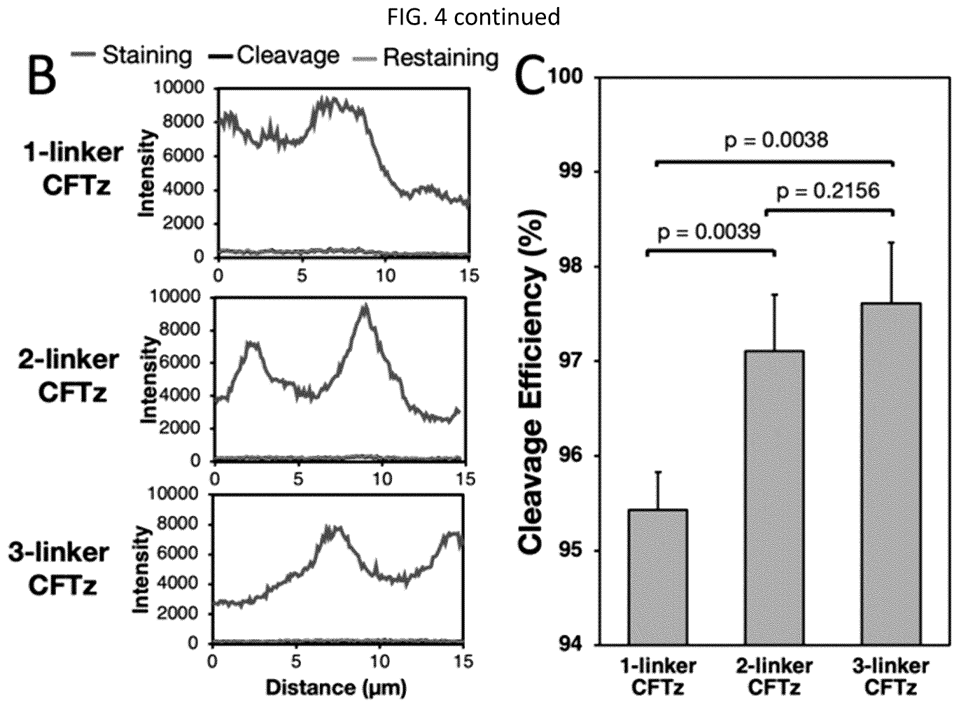

The tetrazine-(N₃)ₙ-Cy5 of Formula III was designed and synthesized (FIG. 2) by tethering fluorophore Cy5 to tetrazine through an azide-based cleavable linker. As FIG. 4C demonstrates, the cleavage efficiency increases with more cleavable centers within the cleavable linker. With two linkers, the cleaving step (i.e., removing the detectable marker from the tetrazine) may be able to remove 97.5% of the detectable marker. With three linkers, the cleaving step may be able to remove about 98% of the detectable marker. The number of cleavable centers may negatively or positively impact the emission of the detectable marker provided by the cleavable detectably-labeled tetrazine. For example, FIG. 4C demonstrates a decrease in signal intensity of the detectable marker when three linkers are used. The synthesis and characterization of tetrazine-(N₃)ₙ-Cy5 is described in Example 1.

Any appropriate detectable label can be used to produce a cleavable detectably-labeled tetrazine. In some embodiments, the detectable label of the cleavable detectably-labeled tetrazine is a fluorophore. In such cases, the cleavable detectably-labeled tetrazine is cleavable fluorescent tetrazine (CFTet). Fluorophores useful in the methods of this disclosure include, without limitation, Cy5, TAMRA (labeled with tetramethylrhodamine or “TMR”), ALEXA FLUOR™ 594, and ATTO 647N and ATTO 700 fluorophores (ATTO-TEC, Germany). Other fluorophores appropriate for use according to the methods provided herein include, without limitation, quantum dots, ALEXA FLUOR™ 350, ALEXA FLUOR™ 532, ALEXA FLUO^{R™} 546, ALEXA FLUOR™ 568, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, DYLIGHT™ DYES (e.g., DYLIGHT™ 405, DYLIGHT™ 488, DYLIGHT™ 549, DYLIGHT™ 594, DYLIGHT™ 633, DYLIGHT™ 649, DYLIGHT™ 680, DYLIGHT™ 750, DYLIGHT™ 800 and the like), Texas Red, and Cy2, Cy3.5, Cy5.5, and Cy7, and sulfonated Cy2, Cy3.5, Cy5, Cy5.5, and Cy7. In addition to the use of fluorophores as a detectable moiety, other labels such as luminescent agents (e.g., chemiluminescent agents), fluorescent proteins, and radioisotopes can also be used as detection tags.

In another aspect, provided herein is a method for multiplexed in situ analysis of biomolecules in a tissue. As used herein, the term “multiplexed” refers to the detection of multiple signals (e.g., two or more signals), such as, for example, analytes, fluorescent signals, analog or digital signals, that are combined into one signal over a shared medium. The term encompasses the detection of multiple signals simultaneously in a single sample or single reaction vessel, as well as the combining of images of multiple signals to obtain one image that reflects the combination.

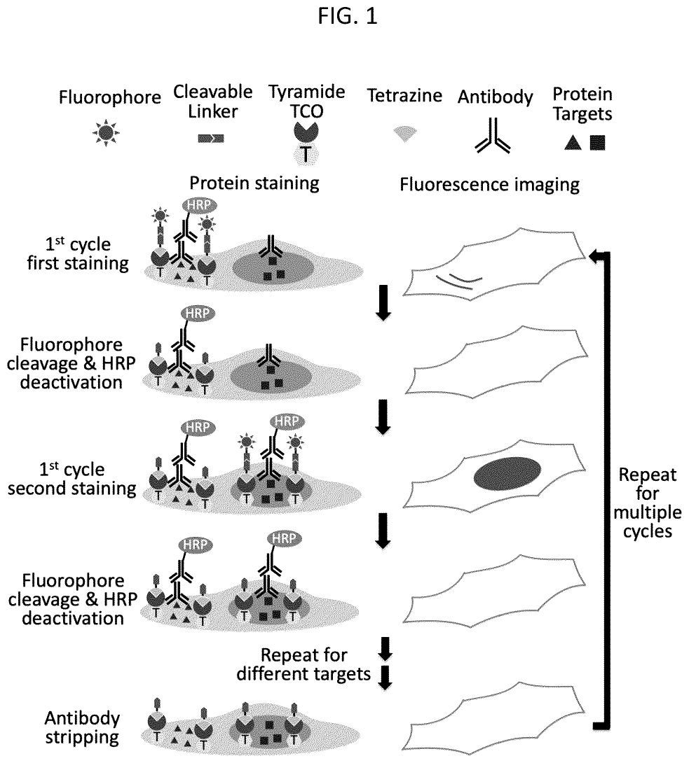

Referring to FIG. 1, the method may include at least three steps in each analysis cycle. First, for analysis of a protein in a tissue, the tissue is contacted with a horseradish peroxidase (HRP)-conjugated antibody configured to bind specifically to the protein target. For analysis of nucleic acids in a tissue, the tissue is contacted with a HRP-conjugated oligonucleotide probe configured to hybridize to the nucleic acid target. In some embodiments, the HRP-conjugated targeting agent is configured to bind directly to the target biomolecule of interest. In some embodiments, the HRP-conjugated targeting agent binds to an intermediate, e.g., binds to a primary antibody or a primary oligonucleotide that binds directly to or hybridizes directly to the target biomolecule.

The tissue is also contacted with a first component comprising a tyramide conjugated to a trans-cylooctene (TCO-tyramide). HRP catalyzes the coupling reaction between the cleavable tyramide and tyrosine residues on an endogenous protein target in close proximity. The tissue is further contacted with a second component including a detectable marker, a cleavable linker, and a tetrazine residue (CFTet). The tetrazine residue conjugates to the tyramide through an irreversible and bioorthogonal Dies Alders cycloaddition between the diene of tetrazine and a dienophile of the trans-cyclooctene, a reaction mechanism commonly known in the art as ‘Click’ chemistry. In a further step, the tissues may be further contacted with unlabeled, free tetrazine to bind with any unlabeled, TCO-tyramide which may remain after contacting the tissue with CFTet.

In subsequent step, fluorescence images are captured to generate quantitative protein expression profiles. Finally, detectable labels attached to the CFTet are chemically cleaved in a step that simultaneously deactivates HRP, which allows for initiation of the next analysis cycle. Through reiterative cycles of target staining, fluorescence imaging, fluorophore cleavage, and HRP deactivation, a large number of different target biomolecules with a wide range of expression levels can be quantified in single cells of intact tissues in situ.

In exemplary embodiments, the method comprises (a) contacting the tissue with a plurality of horseradish peroxidase (HRP)-conjugated targeting agents that are configured to specifically bind or hybridize to the target biomolecule in the contacted tissue, wherein the second contacting step occurs under conditions that promote binding or hybridization of the targeting agents to the target biomolecule; (b) contacting the tissue sample with the compound of Formula I under conditions that promote conjugation of the compound to the target biomolecule; (c) contacting the tissue with the cleavable detectably-labeled tetrazine (CFTet) compound of Formula II or Formula III, under conditions that promote conjugation of the cleavable labeled tetrazine to the compound of Formula I; (d) imaging the tissue thereby detecting the detectable marker; (e) contacting the tissue sample with a composition comprising 1,3,5-Triaza-7-phosphaadamantane (PTA) and tris(2-carboxyethyl) phosphine (TCEP) at about 40° C. for about 30 minutes; and (f) repeating steps (a)-(e), as necessary, depending on the number of biomarkers to be detected. An additional step, after step (e) and before step (f), including contacting the tissue sample with a plurality of unlabeled tetrazine thereby blocking unbound compound of Formula I, which may contribute to false positive signal from generating in the subsequent analysis cycles.

The targeting agent will vary depending on the type of target biomolecule. In some cases, the target biomolecule is a protein or peptide. In such cases, the targeting agent will be an antibody that specifically binds to the target protein or peptide. For example, if the target biomolecule is protein Histone deacetylase 2 (HDAC2), the target agents comprise anti-HDAC2 antibodies conjugated to HRP. Antibodies suitable for the methods include, without limitation, polyclonal antibodies, monoclonal antibodies, and antigen-binding fragments thereof. HRP-conjugated antibodies can be used to detect other target biomolecules such as lipids and metabolites.

In other cases, the target biomolecule is a nucleic acid (e.g., DNA, RNA). In such cases, the targeting agent will be an HRP-conjugated oligonucleotide having sequence complementary to the target nucleic acid sequence. Under appropriate conditions, the HRP-oligonucleotide will hybridize to the target nucleic acid sequence. Upon addition of the TCO-tyramide, HRP catalyzes a coupling reaction between the tyramide in close proximity with tyrosine residues on endogenous proteins that are also in close proximity. In some cases, multiple cycles of the method are performed to detect multiple target biomolecules using targeting agents that are HRP-conjugated antibodies, HRP-conjugated oligonucleotides, or a combination thereof.

In some cases, the target biomolecule is a carbohydrate. In such cases, the targeting agent can be a HRP-conjugated lectin that is capable of binding carbohydrate. As used herein, the term “lectin” refers to a protein or glycoprotein that binds to specific carbohydrate structures to form a lectin-carbohydrate complex. The term encompasses lectins derived from animal and plant sources, and which bind carbohydrates by affinity. The term “lectin” as used herein also encompasses glycoproteins and proteins not normally termed lectins but which immunologically bind carbohydrates, such as antibodies, e.g., monoclonal antibodies. Since lectins bind selectively to some but not all carbohydrates (e.g., monosaccharides, such as mannose, GleNAc, gelatose, a-fructose or sialic acid) to different degrees, it will be understood that the type of lectin conjugated to HRP will vary depending on the target carbohydrate of interest. Upon addition of the TCO-Tyramide, HRP catalyzes a coupling reaction between the tyramide in close proximity with tyrosine residues on endogenous proteins that are also in close proximity.

Any appropriate method of preparing antibody-horseradish peroxidase conjugates can be used. Exemplary protocols for preparation of an HRP antibody conjugate are known in the art. By way of non-limiting example, HRP can be activated for conjugation by treatment with a 100-fold molar excess of a bifunctional PEG linker having a maleimide group and an active ester group. Antibodies to a protein of interest can be prepared for conjugation by introducing thiols using, for example, DTT. A thiolated antibody can be contacted to a molar excess of HRP comprising a bifunctional PEG linker for conjugation.

Likewise, methods of preparing an oligonucleotide probe conjugated to HRP are well known in the art and can be commercially obtained.

In some cases, the HRP-conjugated detection agent (e.g., antibody, oligonucleotide) and TCO-Tyraminde are contacted in the presence of a tyramide signal amplification buffer. In some embodiments, the amplification buffer comprises an aqueous phosphate-buffered, borate-buffered, or other buffered solution to which low concentrations of hydrogen peroxide are added. In some embodiments, the amplification buffer comprises 0.0015% H₂O₂ and 0.1% triton X-100 in 0.1 M boric acid, pH=8.5. Commercial tyramide signal amplification buffers are available from several manufacturers including, for example, PerkinElmer, ThermoFisher, and Biotium.

In some embodiments, the signal from the detectable marker is removed in a “removing step.” In some embodiments, the removing step comprises chemically cleaving the detectable label. Any appropriate means of removing a detectable signal or detectable label (e.g., a fluorophore) can be used according to the methods provided herein. Methods of removal can include without limitation one or more of photobleaching, chemical deactivation, chemical cleavage of the fluorophores (see the Examples below), enzymatic cleavage of the fluorophores, DNA/RNA strand displacement, chemical or heat denaturing of an intermediate fluorescent oligonucleotide, and the like. Since photobleaching can be a time-consuming step, in some cases the methods provided herein comprise efficiently removing fluorescence signals by chemical deactivation or chemical or enzymatic cleavage of detectable labels.

In some embodiments, the methods provided herein comprise chemical inactivation of fluorophores. For example, fluorophores can be inactivated by oxidation. Protocols for oxidation of dyes with hydrogen peroxide, which can be catalyzed using either acidic or basic conditions, or reactive oxygen species (ROS) are known to those practitioners in the art for changing the fluorescent properties of dyes and fluorescent proteins.

In some embodiments, the detectable marker is removed by chemical cleavage of the linker joining the tetrazine and the detectable marker. In some embodiments, the linker is cleaved by contacting the sample with tris(2-carboxyethyl) phosphine (TCEP), and 1,3,5-Triaza-7-phosphaadamantane (PTA), either as separate components or as a composition. In some embodiments, the contacted sample is incubated at a temperature of about 30° C., 31°, C32°, 33° C., 34° C., 35° C., 36° C., C37° C., 38° C., 39° C., C40° C., 41° C., 42° C., C43°, 44° C., 45° C., 46° C., 47° C., C48° C., 49° C. or about 50° C. The time of incubation is about 10 to about 120 minutes, 20 to about 90 minutes, 30 to about 60 minutes, or about 30 minutes. In some embodiments, the sample is washed prior to the next cycle.

When the detectable marker of the CFTet molecule is a fluorophore, fluorescence photomicroscopy can be used to detect and record the results of consecutive in situ analysis using routine methods known in the art. Alternatively, digital (computer implemented) fluorescence microscopy with image-processing capability may be used. Two well-known systems for imaging FISH of chromosomes having multiple colored labels bound thereto include multiplex-FISH (M-FISH) and spectral karyotyping (SKY). See Schrock et al. (1996) Science 273:494; Roberts et al. (1999) Genes Chrom. Cancer 25:241; Fransz et al. (2002) Proc. Natl. Acad. Sci. USA 99:14584; Bayani et al. (2004) Curr. Protocol. Cell Biol. 22.5.1-22.5.25; Danilova et al. (2008) Chromosoma 117:345; U.S. Pat. No. 6,066,459; and FISH TAG™ DNA Multicolor Kit instructions (Molecular probes) for a review of methods for painting chromosomes and detecting painted chromosomes.

To minimize issues of autofluorescence or background signal, oligonucleotide targeting agents can be designed to hybridize to a target nucleic acid at multiple places on the target nucleic acid sequence. Thus, an increased number of oligonucleotides will hybridize to each target nucleic acid sequence (e.g., transcript) to enhance signal to noise ratio. As used herein, the terms “binding,” “to bind,” “binds,” “bound,” or any derivation thereof refers to any stable, rather than transient, chemical bond between two or more molecules, including, but not limited to, covalent bonding, ionic bonding, and hydrogen bonding. The term “binding” encompasses interactions between polypeptides, for example, an antibody and its epitope on a target protein. The term also encompasses interactions between a nucleic acid molecule and another entity such as a nucleic acid or probe element. Specifically, binding, in certain embodiments, includes the hybridization of nucleic acids. In some cases, the methods further comprise a blocking step to reduce background signal. The term “blocking” as used herein refers to treatment of a sample with a composition that prevents the non-specific binding of the target substance to the sample. Typically a blocking composition comprises a protein, such as casein or albumin, and may additionally comprise surfactants. The function of the blocking protein is to bind to the sample to prevent the non-specific binding of assay reagents.

In some cases, the method further comprises a washing step. For example, the method can further comprise washing to remove unhybridized targeting agents and non-specifically hybridized targeting agents prior to the addition of TCO-tyramide to the sample (e.g., prior to the tyramide-HRP reaction) and/or prior to the addition of CFTet, and again prior to visualization. In some embodiments, the methods comprises a washing step after cleaving the detectable label and prior to the next cycle including the addition of a next targeting agent. There may be further washing steps before and/or after contacting the tissue sample with unlabeled tetrazine including contacting the tissue sample with a plurality of unlabeled tetrazine thereby blocking unbound compound of TCO-tyramide, which may contribute to false positive signal from generating in the subsequent analysis cycles.

The methods of this disclosure can be performed using a tissue sample obtained from any biological entity. The term “biological entity” as used herein means any independent organism or thing, alive or dead, containing genetic material (e.g., nucleic acid) that is capable of replicating either alone or with the assistance of another organism or cell. Sources for nucleic acid-containing biological entities include, without limitation, an organism or organisms including a cell or cells, bacteria, yeast, fungi, algae, viruses, or a sample thereof. Specifically, an organism of the current disclosure includes bacteria, algae, viruses, fungi, and mammals (e.g., humans, non-human mammals). The methods and compositions described herein can be performed using a variety of biological or clinical samples comprising cells that are in any (or all) stage(s) of the cell cycle (e.g., mitosis, meiosis, interphase, G0, G1, S and/or G2). As used herein, the term “sample” include all types of cell culture, animal or plant tissue, peripheral blood lymphocytes, buccal smears, touch preparations prepared from uncultured primary tumors, cancer cells, bone marrow, cells obtained from biopsy or cells in bodily fluids (e.g., blood, urine, sputum and the like), cells from amniotic fluid, cells from maternal blood (e.g., fetal cells), cells from testis and ovary, and the like. In some cases, samples are obtained by swabbing, washing, or otherwise collecting biological material from a non-biological object such as a medical device, medical instrument, handrail, door knob, etc. Samples are prepared for assays of this disclosure using conventional techniques, which typically depend on the source from which a sample or specimen is taken. These examples are not to be construed as limiting the sample types applicable to the methods and/or compositions described herein.

In some embodiments, the methods provided herein comprise a cell or tissue fixation step. For example, the cells of a biological sample (e.g., tissue sample) can be fixed (e.g., using formalin, formaldehyde, or paraformaldehyde fixation techniques known to one of ordinary skill in the art). In some cases, the tissue is formalin-fixed and paraffin-embedded (FFPE). Any fixative that does not affect antibody binding or nucleic acid hybridization can be utilized in according to the methods provided herein. In other cases, the methods are performed on unfixed (“fresh”) tissue samples.

As described herein, the methods of the present invention provide for multiplexed in situ analysis of biomolecules in a tissue. Through consecutive cycles of targeting agent binding/hybridization, fluorescence imaging, and signal removal, different biomolecule species can be identified as fluorescent spots with unique color sequences. In some embodiments, the CFTet's of different cycles, which are used in conjunction with different targeting agents, comprise different labels. For example, in a first cycle, a first targeting agent is hybridized to a first target biomolecule, and a first TCO-tyramide, and a first CFTet comprising a first detectable label is used. In a subsequent cycle, an second targeting agent is hybridized to a second target biomolecule, and a second TCO-tyramide, and a second CFTet comprising a second detectable label is used.

As used herein, the term “biomolecule” or “biological molecule” refers to any molecule that is substantially of biological origin and encompasses proteins, peptides, and nucleic acids. Such molecules may include non-naturally occurring components that mimic a naturally occurring component, e.g., a non-naturally occurring amino acid. The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer. As used herein, the terms “nucleic acid” or “oligonucleotide” refer to and encompass any physical string or collection of monomer units (e.g., nucleotides) that can connect to form a string of nucleotides, including a polymer of nucleotides (e.g., a typical DNA or RNA polymer), peptide nucleic acids (PNAs), modified oligonucleotides (e.g., oligonucleotides comprising nucleotides that are not typical to biological RNA or DNA, such as 2′-O-methylated oligonucleotides), and the like. The nucleotides of the nucleic acid can be deoxyribonucleotides, ribonucleotides or nucleotide analogs, and can be natural or non-natural, and can be unsubstituted, unmodified, substituted or modified. The nucleotides can be linked by phosphodiester bonds, or by phosphorothioate linkages, methylphosphonate linkages, boranophosphate linkages, or the like. The nucleic acid can additionally comprise non-nucleotide elements such as labels, quenchers, blocking groups, or the like. The nucleic acid can be single-stranded or double-stranded.

As used herein, the terms “nucleic acid of interest,” and “target nucleic acid” include a nucleic acid originating from one or more biological entities within a sample. The target nucleic acid of interest to be detected in a sample can be a sequence or a subsequence from DNA, such as nuclear or mitochondrial DNA, or cDNA that is reverse transcribed from RNA in the sample. The sequence of interest can also be from RNA, such as mRNA, rRNA, tRNA, miRNA, siRNAs, antisense RNAs, or long noncoding RNAs. More generally, the sequences of interest can be selected from any combination of sequences or subsequences in the genome or transcriptome of a species or an environment. In some cases, a defined set of targeting agents are oligonucleotide probes that are designed to hybridize to the plurality of sequences that would be expected in a sample, for example a genome or transcriptome, or a smaller set when the sequences are known and well-characterized, such as from an artificial source.

Oligonucleotide probes useful for the methods provided herein are of any length sufficient to permit probe penetration and to optimize hybridization of probes for in situ analysis according to the methods of this disclosure. Preferably, probe length is about 20 bases to about 500 bases. As probe length increases, so increases the number of binding sites that can be incorporated into a given probe for hybridization to the probe of the following cycle as well as the signal to noise ratio. However, longer than 500 bases, the probes may not efficiently penetrate the cellular membrane. Preferably, the oligonucleotide probes have a probe length between 20 and 500 nucleotides, 20 and 250, 50 and 250, 150 and 250 nucleotides, 20 and 150, or 50 and 150 nucleotides, inclusive.

The terms “hybridize” and “hybridization” as used herein refer to the association of two nucleic acids to form a stable duplex. Nucleic acids hybridize due to a variety of well characterized physico-chemical forces, such as hydrogen bonding, solvent exclusion, base stacking and the like. An extensive guide to the hybridization of nucleic acids is found in Tijssen (1993) Laboratory Techniques in Biochemistry and Molecular Biology-Hybridization with Nucleic Acid Probes, part I chapter 2, “Overview of principles of hybridization and the strategy of nucleic acid probe assays” (Elsevier, N.Y.). One of skill in the art will understand that “hybridization” as used herein does not require a precise base-for-base complementarity. That is, a duplex can form, between two nucleic acids that contained mismatched base pairs. The conditions under which nucleic acids that are perfectly complementary or that contain mismatched base pairs will hybridize to form a duplex are well known in the art and are described, for example, in MOLECULAR CLONING: A LABORATORY MANUAL, 3^{rd} ed., Sambrook et al., eds., Cold Spring Harbor Press, Cold Spring Harbor (2001) at Chapter 10, which is herein incorporated by reference. As used herein, the term “complementary” refers to a nucleic acid that forms a stable duplex with its “complement”. For example, nucleotide sequences that are complementary to each other have mismatches at less than 20% of the bases, at less than about 10% of the bases, preferably at less than about 5% of the bases, and more preferably have no mismatches.

### Kits

In another aspect, provided herein is a kit comprising reagents for performing multiplexed in situ analysis of biomolecules in a tissue. Preferably, the kit comprises a TCO-Tyramide of Formula I, a cleavable detectably-labeled tetrazine of Formula II or III, and a written insert component comprising instructions for performing multiplexed in situ analysis of target biomolecules according to the methods provided herein. In some embodiments, the kit further comprises a one or more HRP-conjugated targeting agents configured to bind or hybridize to a target biomolecule. As described herein, the targeting agents can be synthetic DNA oligonucleotide probes, polyclonal antibodies, monoclonal antibodies, antigen-binding fragments of an antibody, or some combination thereof. In some embodiments, the plurality of HRP-conjugated targeting agents comprises HRP-conjugated synthetic DNA oligonucleotide probes. In some embodiments, the plurality of HRP-conjugated targeting agents comprises HRP-conjugated polyclonal or monoclonal antibodies, or antigen-binding fragments thereof. In some embodiments, the kit further comprises an amplification reaction buffer, a blocking reagent, and/or a hydrogen peroxide additive. The kit may also contain unlabeled tetrazine which may be used to eliminate false-positive labeling in subsequent labeling iterations arising from one or more TCO-tyramide being unlabeled by cleavable detectably-labeled tetrazine in an earlier iteration of labeling and imaging.

In some embodiments, the detectable maker includes, without limitation, fluorophores, luminescent agents (e.g., chemiluminescent agents), fluorescent proteins, and radioisotopes. By way of example, detectable markers include Cy5, sulfonated cy5, TAMRA (labeled with tetramethylrhodamine or “TMR”), ALEXA FLUOR™ 594, and ATTO 647N and ATTO 700 fluorophores (ATTO-TEC, Germany). Other fluorophores appropriate for use according to the compositions and methods provided herein include, without limitation, quantum dots, ALEXA FLUOR™ 350, ALEXA FLUOR™ 532, ALEXA FLUOR™ 546, ALEXA FLUOR™ 568, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, DYLIGHT™ DYES (e.g., DYLIGHT™ 405, DYLIGHT™ 488, DYLIGHT™ 549, DYLIGHT™ 594, DYLIGHT™ 633, DYLIGHT™ 649, DYLIGHT™ 680, DYLIGHT™ 750, DYLIGHT™ 800 and the like), Texas Red, and Cy2, Cy3.5, Cy5.5, and Cy7, and sulfonated Cy2, Cy3.5, Cy5, Cy5.5, and Cy7. In some embodiments, the detectable marker is a sulfonated Cy 5, and in some embodiments, there is provided a cleavable detectably-labeled tetrazine (CFTet) comprising the compound of Formula (II).

In some embodiments, the kit includes instructions for employing the kit components as well the use of any other reagent not included in the kit. Instructions may include variations that can be implemented. In some embodiments, the kit further comprises tris(2-carboxyethyl) phosphine (TCEP) and 1,3,5-Triaza-7-phosphaadamantane (PTA) either as separate components or as a composition. In such cases, the written instruction component further comprises instructions for removing the detectable label from the detectably-labeled tetrazine using the TCEP/PTA.

### Miscellaneous

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference for the purpose of describing and disclosing the chemicals, cell lines, vectors, animals, instruments, statistical analysis and methodologies which are reported in the publications which might be used in connection with the invention. Nothing herein is to be construed as an admission that the invention is not entitled to antedate such disclosure by virtue of prior invention.

As used herein and in the appended claims, the singular forms “a”, “an” and “the” include plural reference, unless the context clearly dictates otherwise. Thus, for example, reference to “a cell” includes a plurality of such cells and equivalents thereof known to those skilled in the art, and so forth. As well, the terms “a” (or “an”), “one or more” and “at least one” can be used interchangeably herein. In addition, the terms “comprising”, “including” and “having” can be used interchangeably.

As used herein, “about” means within 5% of a stated concentration range or within 5% of a stated time frame.

Various embodiments and aspects of the present invention as delineated hereinabove and as claimed in the claims section below find experimental support in the following examples which, together with the above descriptions, illustrate some embodiments of the invention in a non-limiting fashion.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Example 1

One common feature of complex biological systems, such as brain tissues or solid tumors, is that they are composed of many molecularly and functionally different cell types. Single-cell in situ proteomic technologies are powerful tools to study the regulation, function, and interaction of the varied cell types in these heterogeneous biological systems. Mass spectrometry and microarray technologies have wide applications for comprehensive protein analysis. However, as these approaches require the proteins to be isolated from their original cellular context, the spatial information of the proteins is lost during analysis. Immunofluorescence allows proteins to be quantified in their original cellular contexts. However, due to the spectral overlap of the common fluorophores, only a handful of proteins can be visualized in one specimen by immunofluorescence.

To enable single-cell spatial proteomic analysis, cyclic immunofluorescence and mass spectrometry imaging have been developed. Although these approaches allow a large number of proteins to be quantified in their native cellular contexts with the subcellular resolution, some nonideal factors still exist. For instance, mass spectrometry imaging suffers from long assay time and low sample throughput, as it analyzes the specimen pixel by pixel. In cyclic direct immunofluorescence, primary antibodies are labeled with fluorophores. Without further signal amplification, the detection sensitivity of these technologies can be limited, which hinders their applications to profile low expression proteins or to study specimen with high autofluorescence. Other methods amplify the staining signals using primary antibodies conjugated with oligonucleotides, haptens, or horseradish peroxidase (HRP). Nonetheless, for many protein targets, such chemically modified primary antibodies are not commercially available. And to prepare and validate a panel of those antibodies can be technically demanding, time consuming and expensive.

To enable highly sensitive and multiplexed protein imaging with unconjugated primary antibodies, our laboratory recently developed a spatial proteomic assay using cleavable fluorescent tyramide (CFT). In this approach, protein targets are labeled with off-the-shelf primary antibodies. Subsequently, HRP conjugated secondary antibodies and CFT are applied to stain the targets. After imaging, the fluorophores are chemically cleaved and the antibodies are stripped. Through reiterative cycles, highly sensitive and multiplexed protein imaging can be achieved. This method applies HRP to catalyze the conversion of CFT to highly reactive and short-lived radicals, which covalently bind to nearby tyrosine residues. Such enzymatic deposition of CFT dramatically increases the signal intensities. The success of this approach may improve when the molecular weight of the CFT to be relatively small, so that the short-lived radicals can diffuse efficiently and conjugate to many tyrosine residues in close proximity. However, it may be desirable to have various moieties with large molecular weights to be incorporated into CFT to further improve its performance. For example, the cleavable linkers with different sizes can be applied to enhance signal removal efficiency and reduce the assay time. In some instances, quantum dots and polyfluorophores may be used to increase the detection sensitivity and multiplexing capacity.

Reported herein is the development of clickable and cleavable fluorophores for ultrasensitive and multiplexed protein imaging. In this approach, instead of depositing cleavable fluorophores directly by HRP, small trans-cyclooctene (TCO) moieties are first conjugated to the tyrosine residues close to the protein targets. Subsequently, click chemistry is applied to couple the cleavable fluorescent tetrazine to TCO by Diels-Alder cycloaddition. Using this approach, Inventors successfully labeled the proteins with bulky cleavable fluorophores with multiple cleavage sites. As a result, the signal removal efficiency is significantly improved to 97.5%. Through reiterative cycles of target staining, fluorescence imaging, and fluorophore cleavage, 28 distinct proteins were profiled in single cells of a human formalin-fixed paraffin-embedded (FFPE) tonsil tissue. Based on the unique protein expression patterns and the microenvironment of the individual cells, Inventors partitioned ˜820,000 cells in the same tissue into different clusters. Additionally, Inventors observed that certain cell clusters were predominantly present in specific regions of the tissue, indicating the existence of distinct cellular neighborhoods within the tonsil tissue.

### Results

### Platform Design

In this ultrasensitive and multiplexed protein imaging method, each analysis cycle consists of six major steps, as shown FIG. 1. First, off-the-shelf primary antibodies from different species or of varied immunoglobulin classes are applied to bind to the protein targets. Second, one protein target is then stained with HRP conjugated primary or secondary antibodies and fluorescently labeled. Third, images are captured under a fluorescence microscope, generating single-cell protein expression profiles. To facilitate image alignment, the nucleus is also stained with DAPI and imaged along with the protein target. Fourth, the fluorophores are cleaved chemically, and HRP is simultaneously deactivated. Fifth, steps two to four are repeated until all protein targets in this analysis cycle are detected. Finally, all antibodies are stripped to initiate the next analysis cycle. By repeating cycles of protein staining, fluorescence imaging, fluorophore removal, HRP deactivation, and antibody stripping, this technology enables ultrasensitive and multiplexed in situ protein profiling in single cells of intact tissues.

To minimize the size of HRP substrates while ensuring the large moieties to be successfully applied for target staining, Inventors split the staining probes into two compartments (FIG. 2). The first compartment is consisted of the tyramide moiety and trans-cyclooctene (TCO); while the second compartment contains the fluorophores tethered to tetrazine through cleavable linkers. The small size of tyramide-TCO allows it to be efficiently deposited by HRP to conjugate with many tyrosine residues proximal to the protein of interest. Subsequently, cleavable fluorescent tetrazine (CFTz) are coupled to the deposited TCO moieties through an irreversible and bioorthogonal Diels Alders cycloaddition. Unlike the direct labeling with short-lived (<1 ms) radicals, this click chemistry reaction enables much longer reaction time (mins to hrs). As a result, the relatively large-sized CFTz can be successfully applied for target staining. To assess the effectiveness of this approach, Inventors designed CFTz with one (Tetrazine-N₃-Cy5), two (Tetrazine-N₃—N₃-Cy5) or three (Tetrazine-N₃—N₃—N₃-Cy5) cleavage sites. As long as one site is cleaved, the fluorescent signals may be successfully erased. Thus, the CFTz with multiple cleavage sites should have higher signal removal efficiency, which could lead to improved multiplexing capacity and analysis accuracy.

Efficient Staining by CFTz with Multiple Cleavage Sites

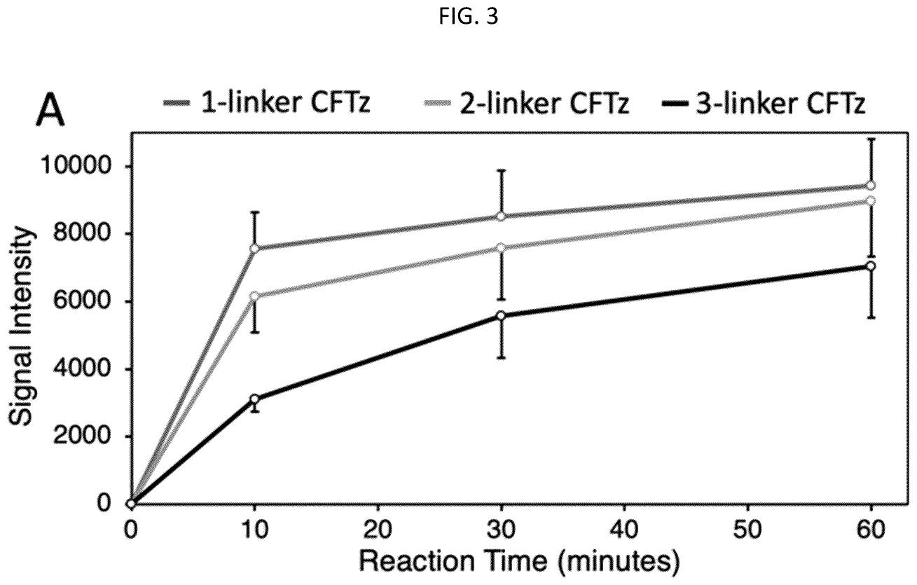

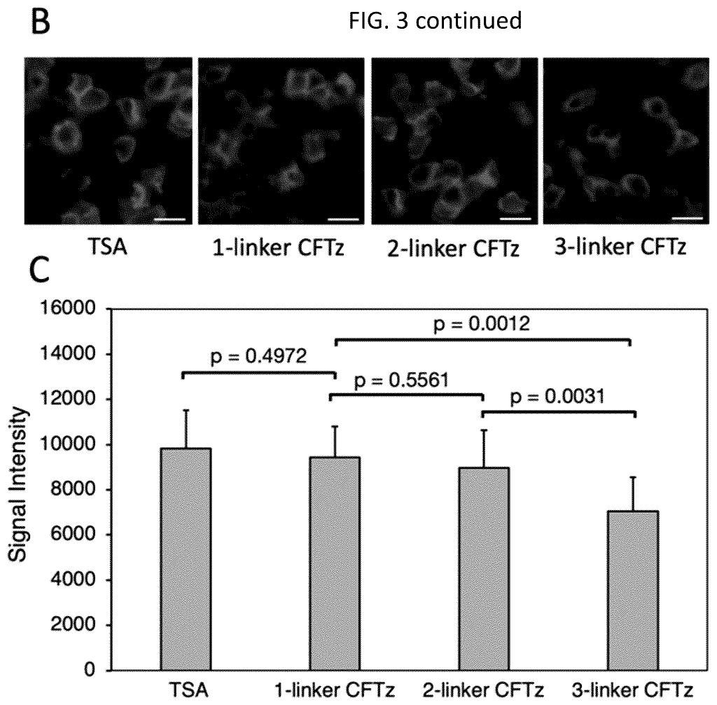

To evaluate whether proteins can be effectively stained by clickable and cleavable CFTz, Inventors labeled protein CD45 in human tonsil FFPE tissues. Due to the fast reaction kinetics of the click chemistry, 1-linker and 2-linker CFTz reached ˜90% and ˜80% of the maximum fluorescence intensity within 10 min, respectively. For 3-linker CFTz, it may need 30 min to obtain ˜80% of the maximum fluorescence intensity (FIG. 3A). The staining patterns obtained with CFTz are consistent with the conventional tyramide signal amplification (TSA) assay (FIG. 3B). And the staining intensities generated by TSA, 1-linker and 2-linker CFTz closely resemble each other; while the intensities obtained by 3-linker CFTz is ˜25% lower (FIG. 3C). These results indicate proteins in FFPE tissues can be efficiently stained by CFTz with a short labeling time.

Enhanced Cleavage Efficiency by CFTz with Multiple Cleavage Sites

To assess the fluorophore cleavage efficiency of CFTz, Inventors stained the protein CD45 and subsequently treated the slides with the mild reducing reagents tris(2-carboxyethyl) phosphine (TCEP) and 1,3,5-Triaza-7-phosphaadamantane (PTA) (FIG. 4A). After cleavage, almost all the fluorescence signals are removed (FIG. 4B). Inventors then quantified and compared the cleavage efficiency of 1-linker, 2-linker, and 3-linker CFTz (FIG. 4C). 1-linker CFTz has the cleavage efficiency of ˜95.5%, which is consistent with previously developed cleavable fluorescent probes. With multiple cleavage sites, 2-linker, and 3-linker CFTz increased the cleavage efficiency to ˜97.5%. By reducing the signal leftover by ˜50% in each analysis cycle, CFTz with multiple cleavage sites will significantly enhance the protein quantification accuracy and the multiplexing capacity of reiterative protein staining approaches.

To ensure that all the unreacted TCO are quenched, Inventors incubated the tissues with free tetrazine following cleavage. Then, Inventors restained the tissues with CFTz, and observed no fluorescence signal increase (FIGS. 4A and 4B). These results suggest that the unreacted TCO are efficiently quenched at the end of each staining cycle. Thus, it will not lead to false positive signals in subsequent protein quantification. Additionally, as documented in previous studies, the TCEP, PTA and antibody stripping treatments will not damage the integrity of epitopes. Therefore, the CFTz with the improved cleavage efficiency should enable a large number of varied proteins to be accurately quantified in single cells in situ.

### Multiplexed In Situ Protein Profiling in the Human Tonsil Tissue

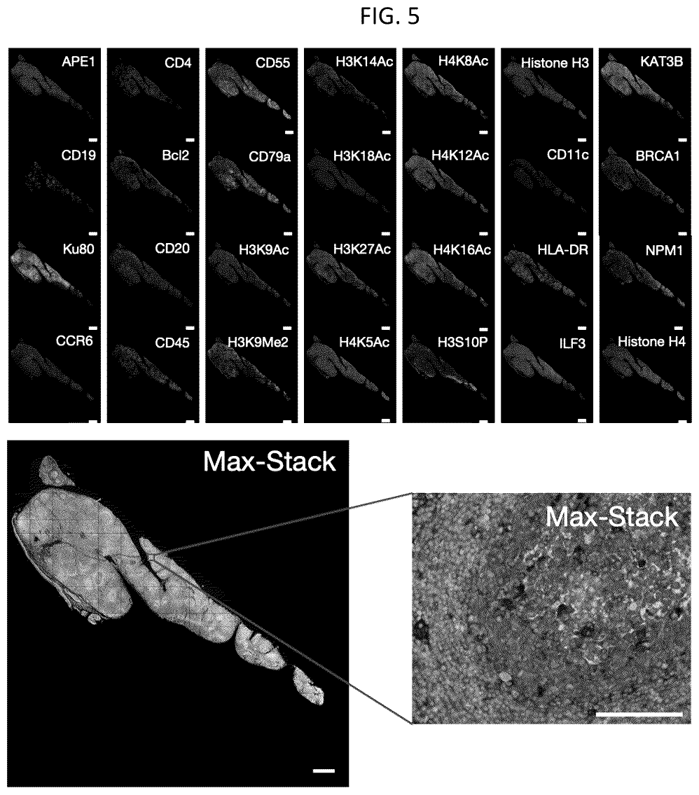

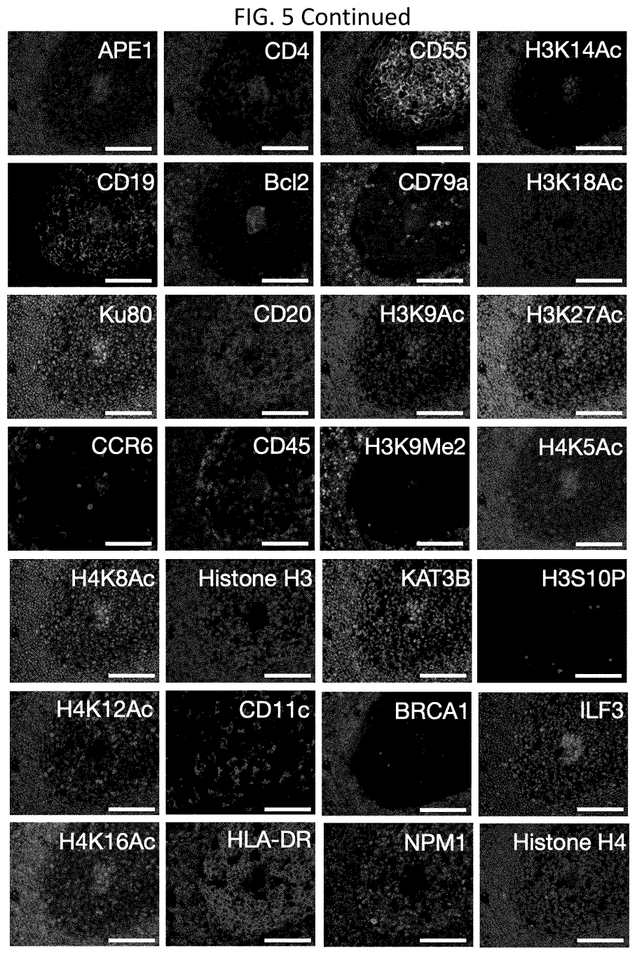

To demonstrate the feasibility of this approach for multiplexed protein imaging, Inventors stained 28 different proteins sequentially on a human FFPE tonsil tissue. Due to its fast reaction kinetics and high cleavage efficiency, 2-linker CFTz (Tetrazine-N₃—N₃-Cy5) was applied to carry out the study. With off-the-shelf antibodies, all the 28 proteins were successfully detected with the subcellular resolution (FIG. 5). The obtained staining patterns are consistent with the ones generated by conventional immunohistochemistry. With the enzymatic signal amplification by HRP, Inventors' approach preserves the super-high sensitivity of the TSA assay. Thus, this approach enables proteins to be unambiguously detected in highly autofluorescent FFPE tissues with millisecond exposure time and a 4× objective. This imaging setup dramatically reduces the assay time and enhances the sample throughput. Additionally, by improving the cleavage efficiency by CFTz with multiple cleavage sites, this method allows more proteins to be precisely quantified in the same specimen. By quantifying 28 proteins in the same tissue, our approach has the highest multiplexing capacity among all the TSA based assays.

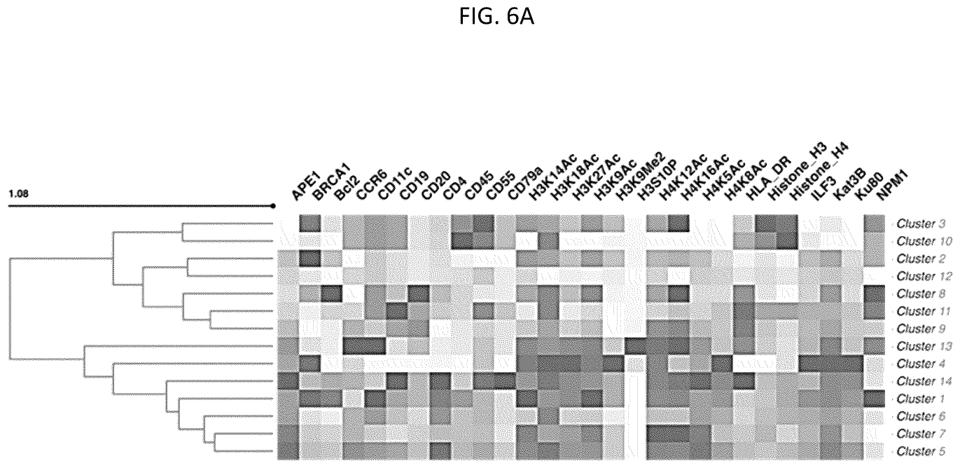

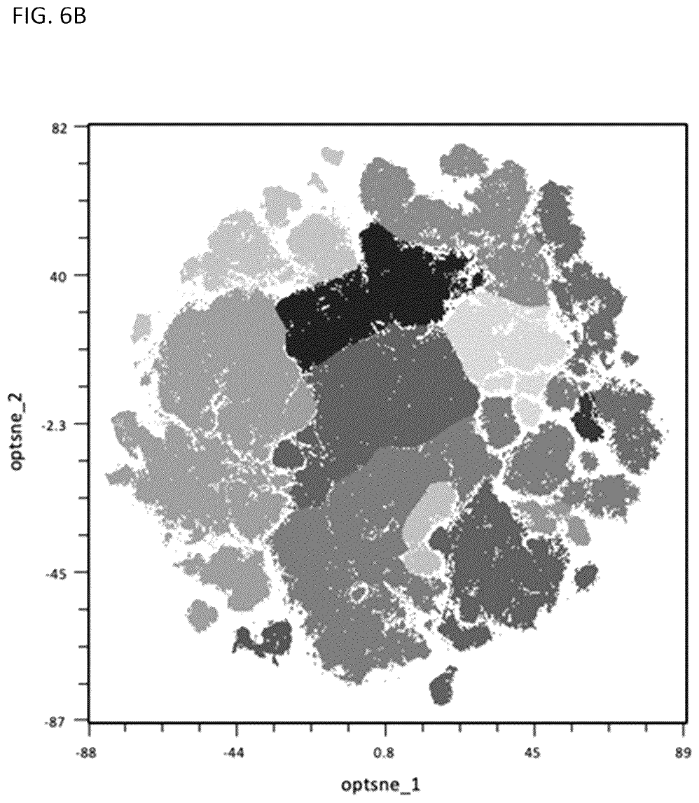

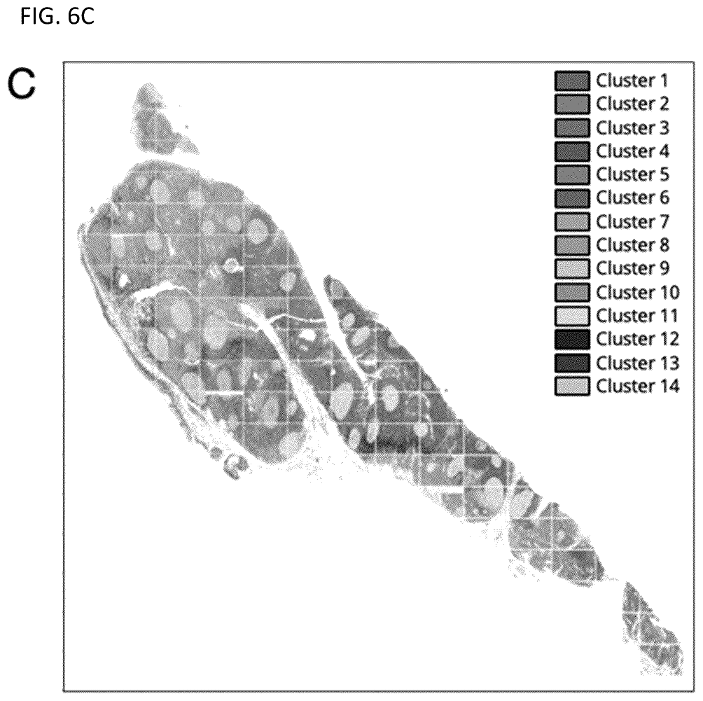

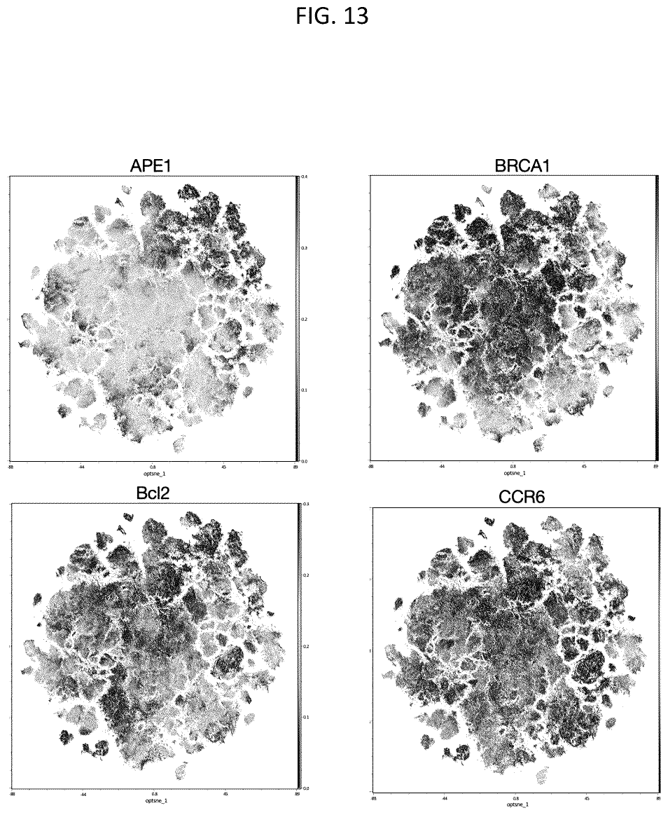

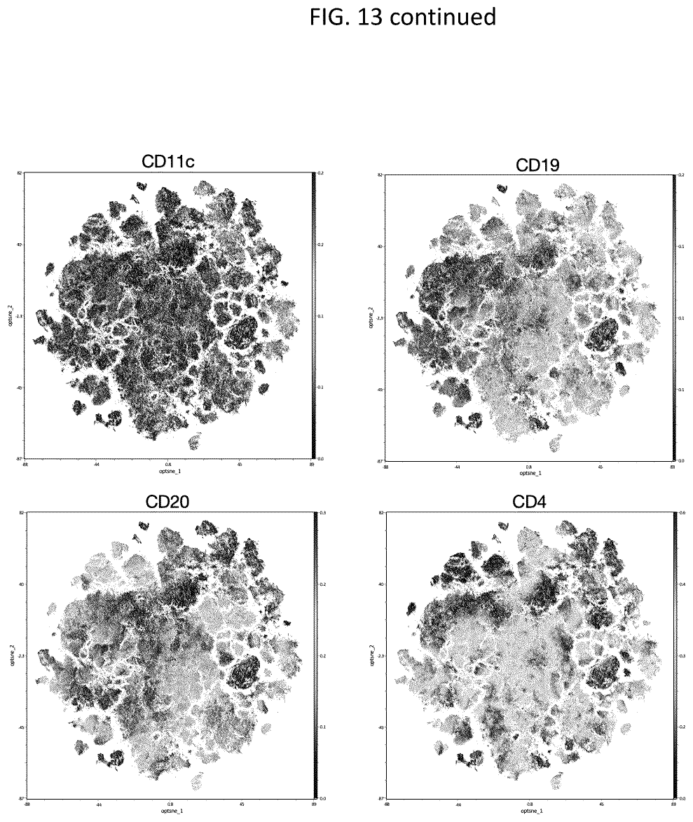

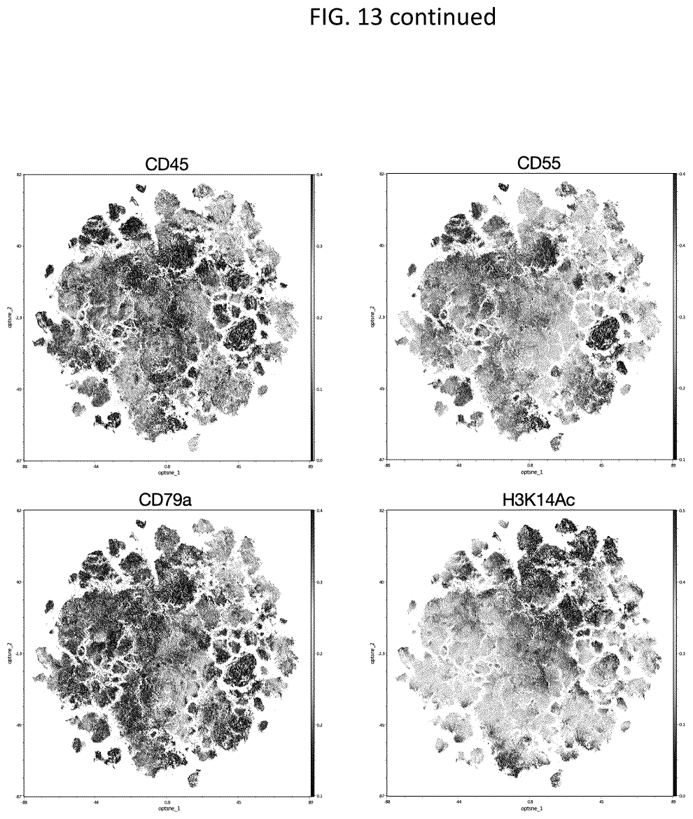

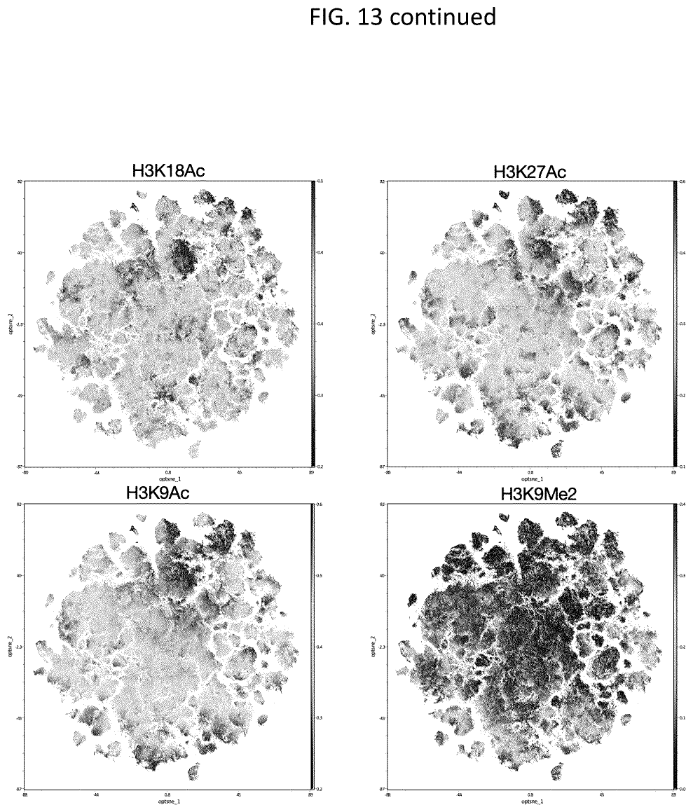

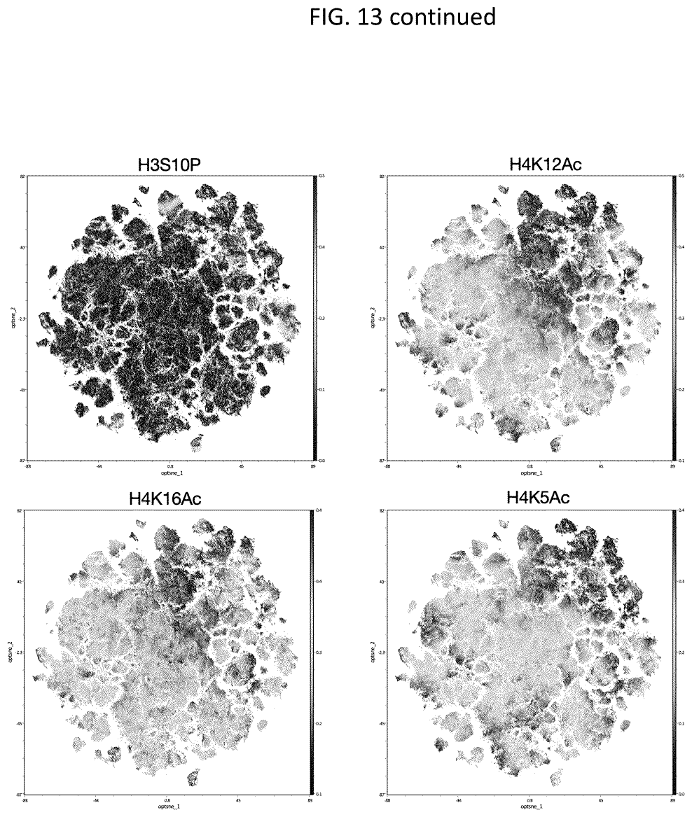

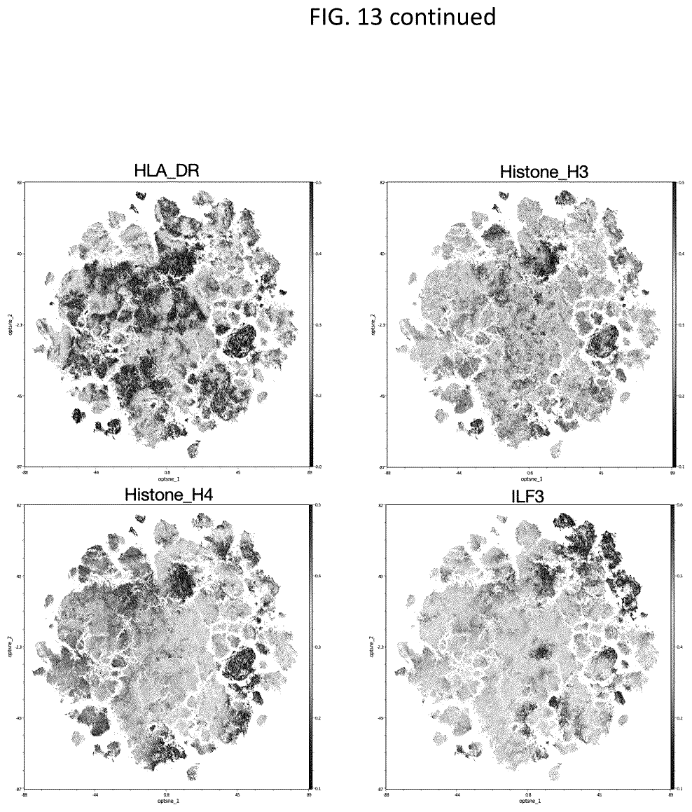

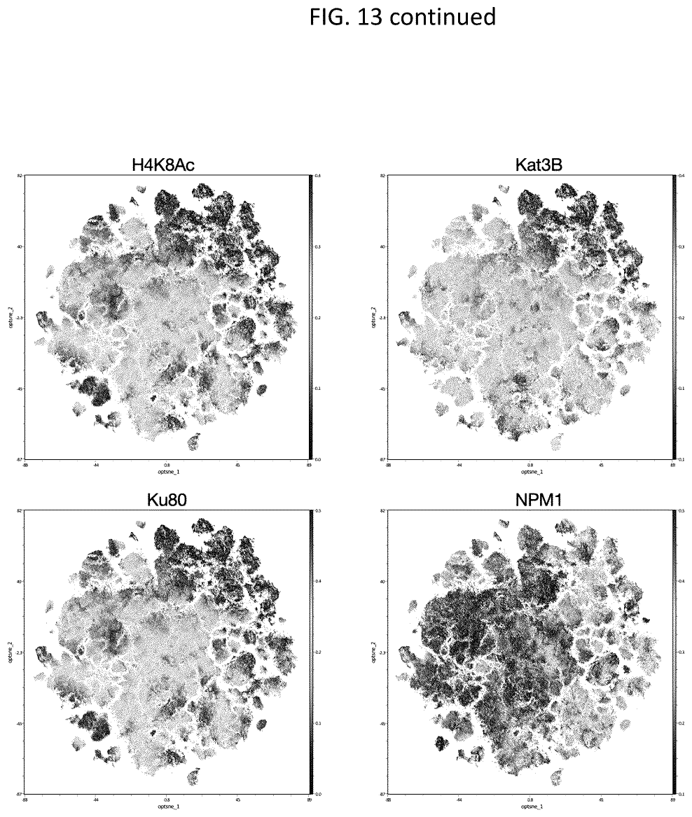

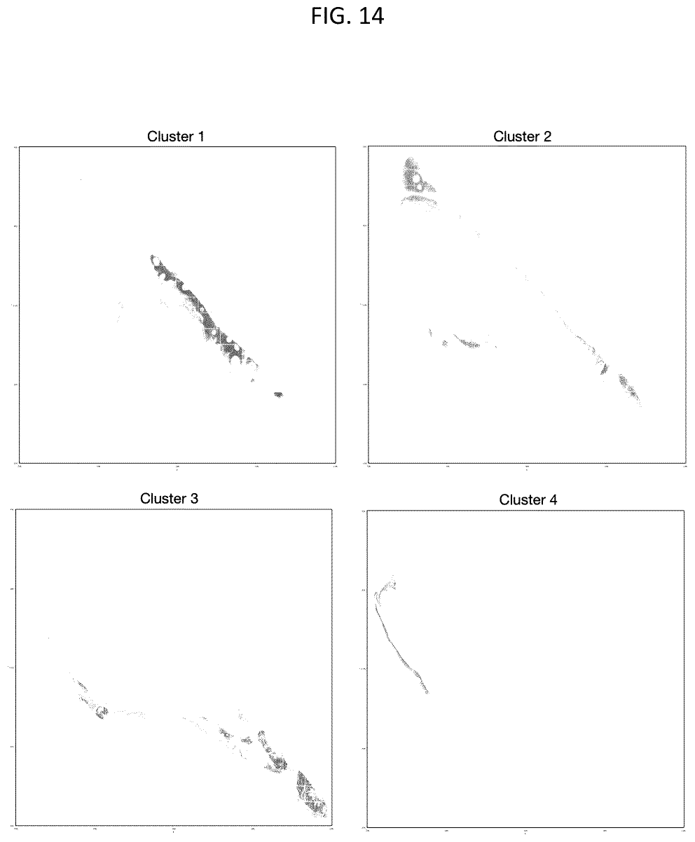

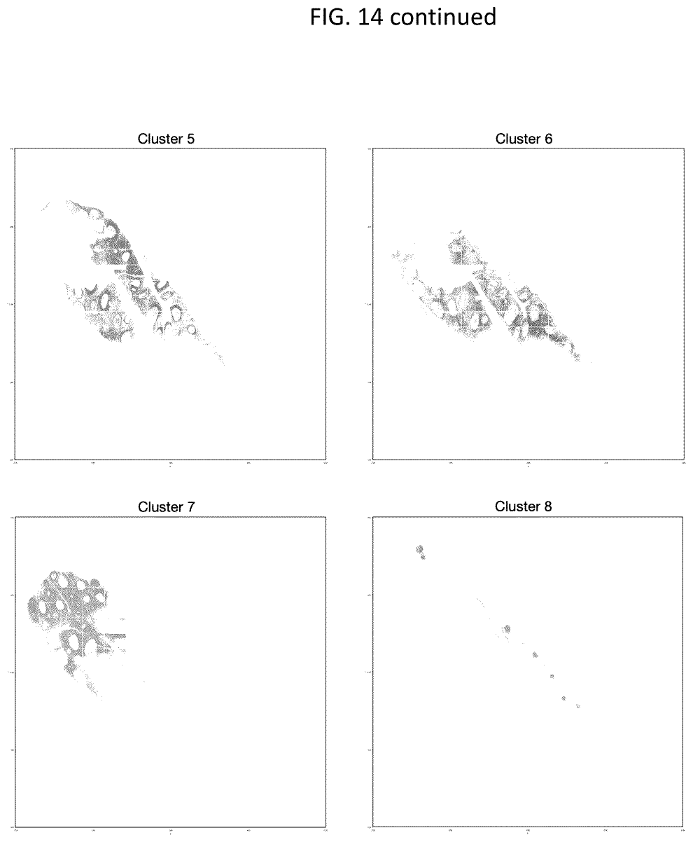

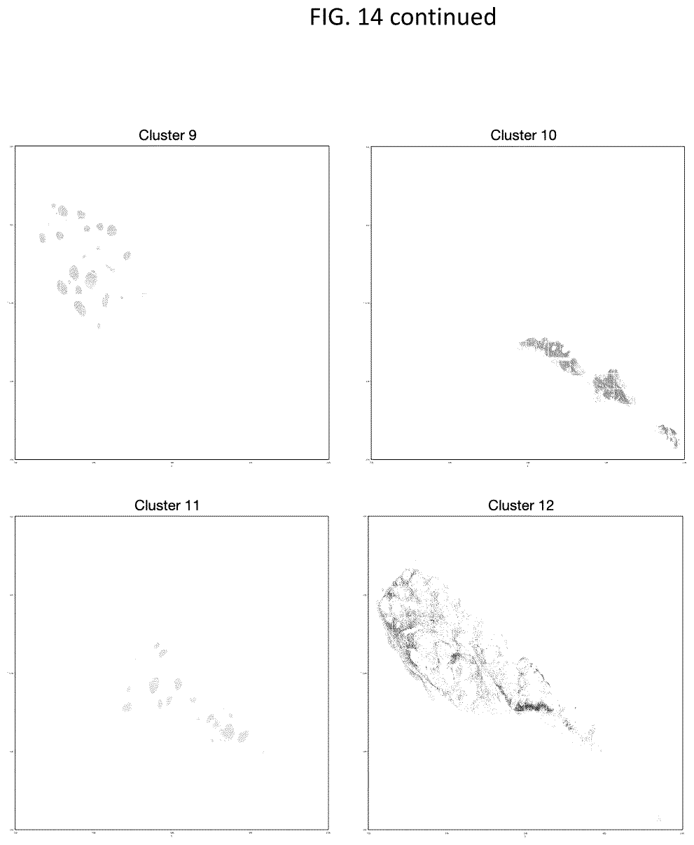

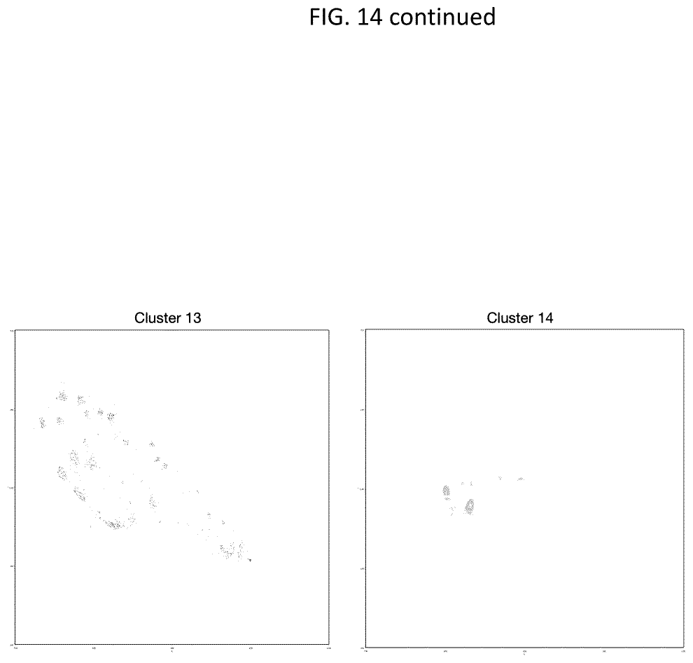

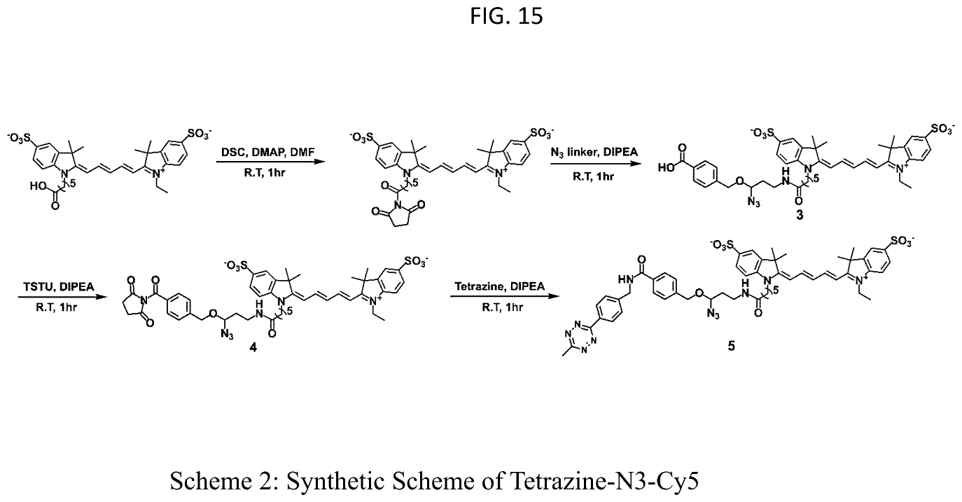

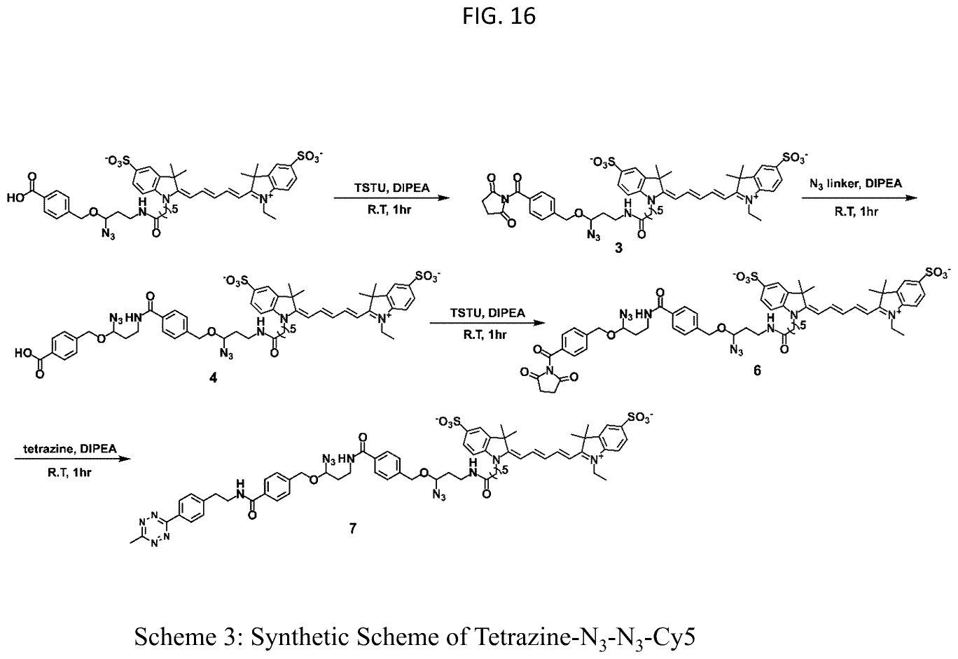

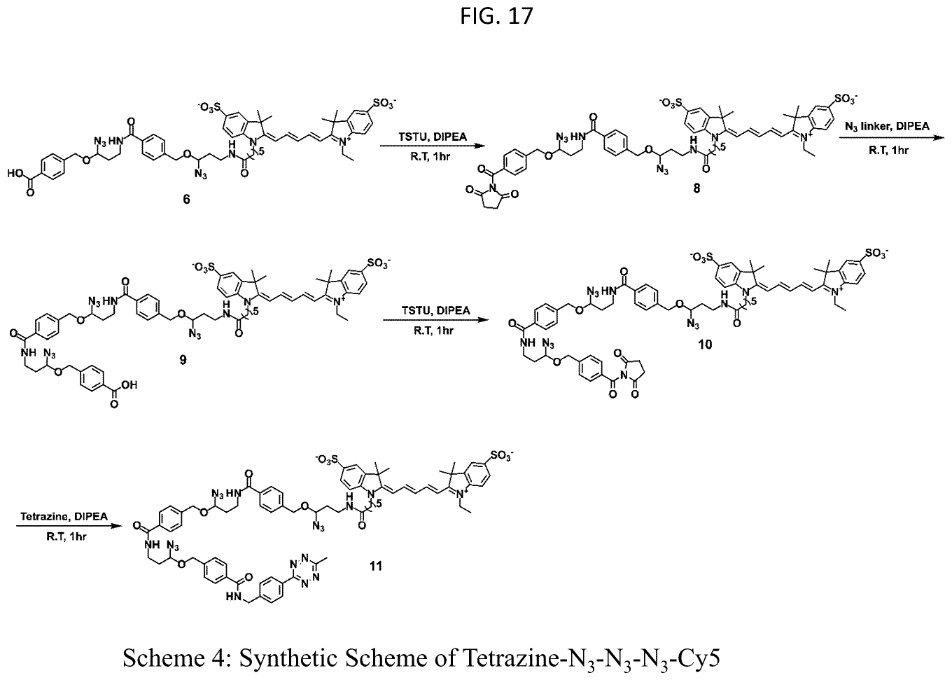

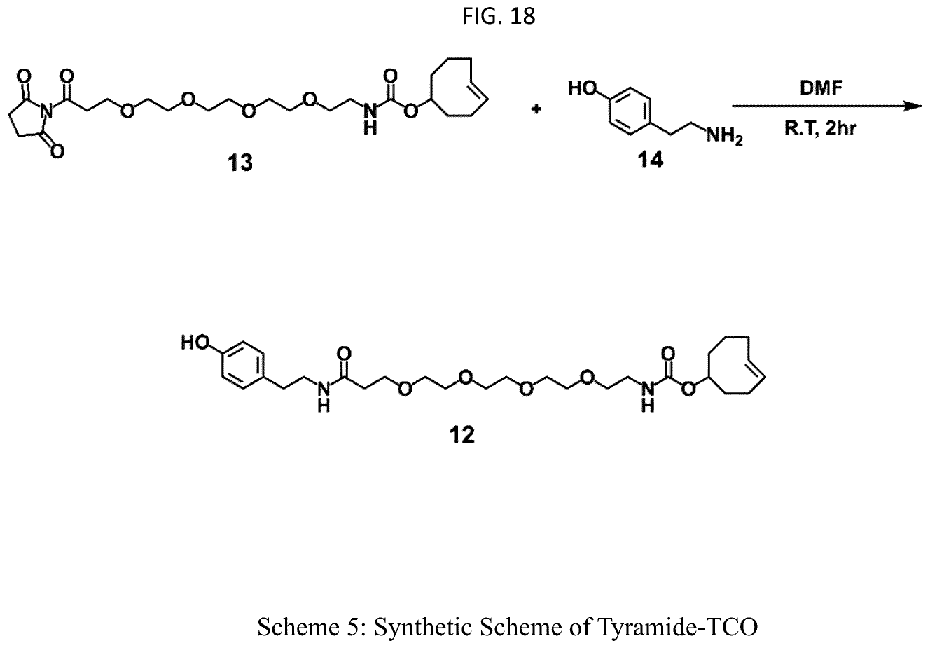

Inventors utilized the single-cell in situ protein expression profiles to investigate the heterogeneity and spatial distribution of various cell types in the human tonsil tissue. To accomplish this, Inventors calculated the expression levels of the 28 proteins in each of the ˜820,000 cells identified in the tissue. Based on their unique protein expression patterns (FIG. 6A and FIG. 13), Inventors partitioned the cells into 14 distinct cell clusters (FIG. 6B) using the Optsne software. These cell clusters were then mapped back to their natural tissue locations (FIG. 6C and FIG. 14), revealing that different subregions of the tonsil tissue were predominantly composed of cells from specific clusters. For example, cluster 4 was mainly present in the epithelium; clusters 2 and 3 dominated the submucosa and connective capsule; clusters 1 and 5 were the major cell type in the lymphoid follicles; clusters 8, 9, 11, 13 and 14 dominated the germinal centers; and clusters 6, 7 and 12 were exclusive to the connective tissues. These results demonstrate that this approach enables the classification of cell types and study of their spatial distribution in formalin-fixed paraffin-embedded (FFPE) tissues.

### Cell-Cell Interaction in Human Tonsil Tissue

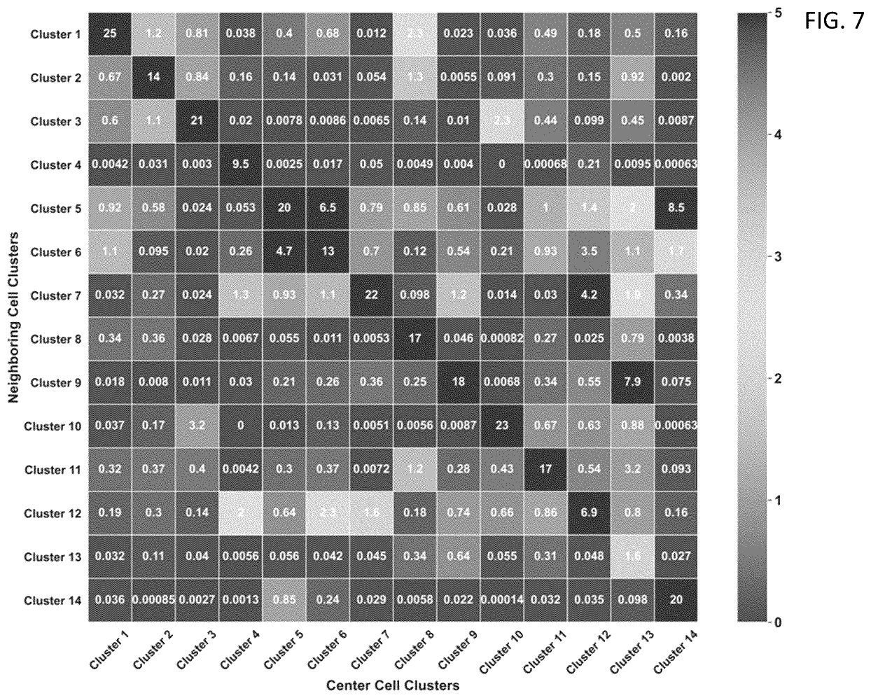

By classifying the cell types in their native spatial contexts, this approach also enables the exploration of the cell-cell interactions among the various cell clusters (FIG. 7). The term “cell neighborhood” was defined as all the cells within the distance of 20 μm from the center cell. For each single cell in the tissue, Inventors counted the number of cells from different clusters in its neighborhood. Then, Inventors calculated the averaged cell numbers of each cluster in varied cell neighborhoods. By projecting the metrics on a heatmap, Inventors observed a significant association between cell clusters 5 and 6, 9 and 13, and 5 and 14. Interestingly, Inventors found a consistently strong association between cells from the same cell cluster (FIG. 7, diagonal), while most of the cells from distinct clusters avoided contact. These results suggest that homotypic cell adhesion may play a significant role in shaping the architecture of human tonsil tissue.

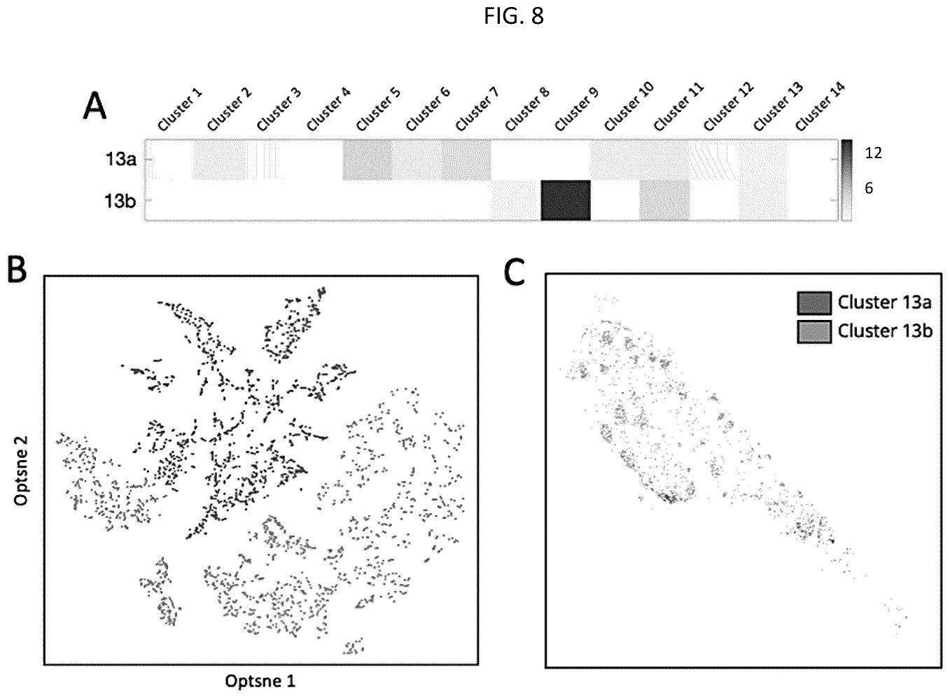

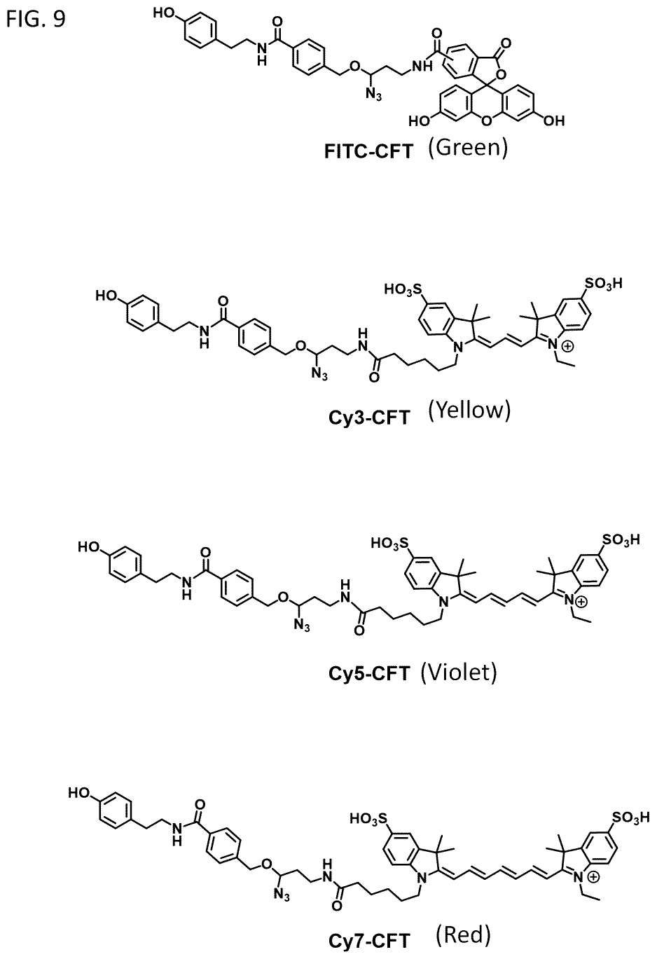

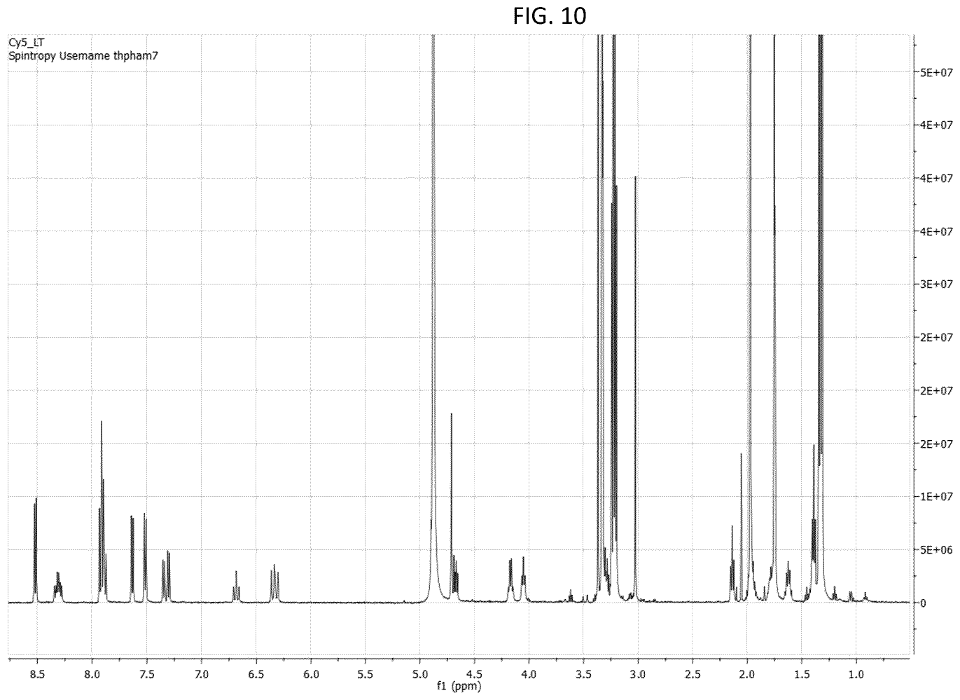

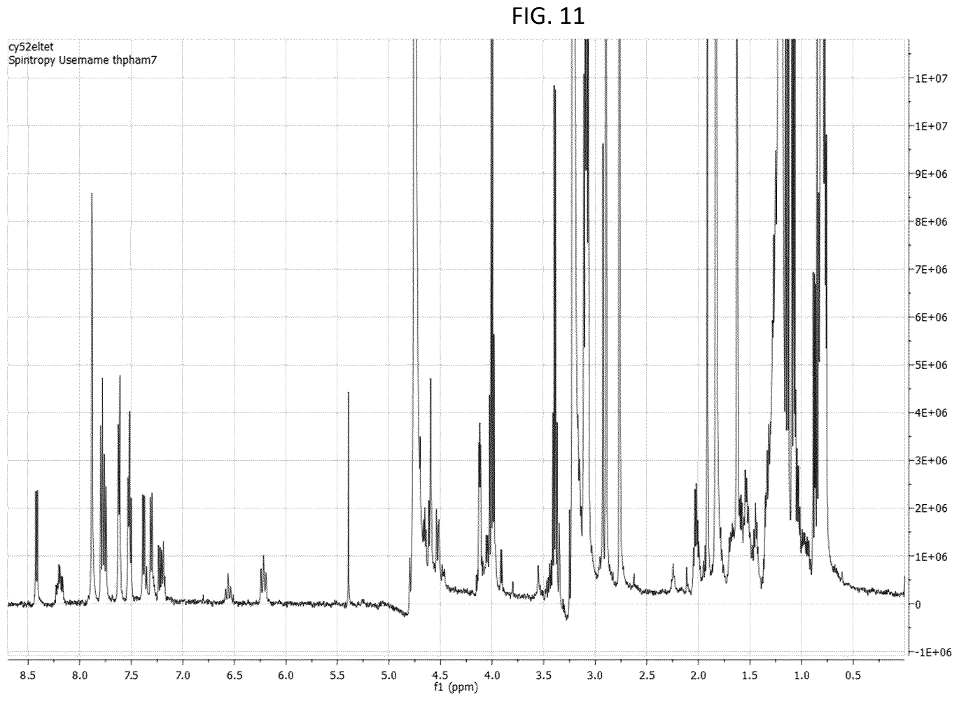

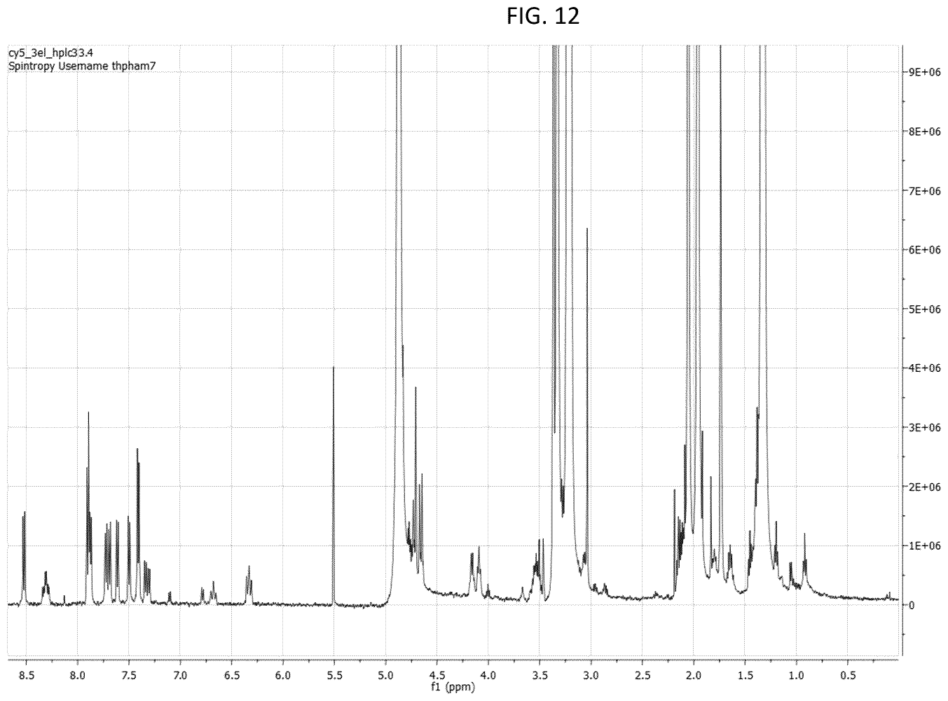

The individual cells from the same cluster can also be further classified into subclusters, based on the cells in their neighborhood. By mapping these subclusters back to their original tissue locations, Inventors observed that varied subclusters from the same cell cluster are located in specific subregions of the tonsil tissue. For instance, cluster 13 was further partitioned into 2 subclusters: 13a and 13b (FIGS. 8A and 8B). Subcluster 13a was predominantly present in the germinal centers; while subcluster 13b was mainly in connective tissues (FIG. 8C). These findings suggest this approach enables the studies of cell-cell interaction and cell type classification based on their microenvironment.

In this study, Inventors have successfully developed clickable and cleavable fluorescent probes, and applied them for highly sensitive and multiplexed single-cell in-situ protein profiling. This approach has overcome the limitation that only the small probes can be applied in the TSA assay. Thus, the bulky fluorophores, such as quantum dots and polyfluorophores, can also be applied for HRP-catalyzed enzymatic target staining. Using this approach, Inventors have designed and synthesized the fluorescent tyramide with multiple cleavage sites. With these probes, the signal removal efficiency is significantly improved, compared to probes with only one cleavage site. As a result, this approach enhances the analysis accuracy and the multiplexing capacity of the existing multiplexed protein imaging technologies.

In this study, primary and secondary antibodies are stripped together by high temperature to enable the subsequent protein analysis. Other methods, including HRP quenching by peroxidase inhibitors (Histochem Cell Biol. 2006 August; 126 (2): 283-291; which is incorporated herein in its entirety and for any purpose), such as H₂O₂, NaN₃, HCl, glucose/glucose oxidase, can also be applied to deactivate HRP to allow the following protein detection. Additionally, primary and/or secondary antibodies can also be stripped by their targets, such as proteins, peptides, IgG, IgA, IgD, IgE, IgM antibodies and small molecules, or digested by enzymes to allow the specific protein staining afterwards.

With this method, Inventors have demonstrated the ability to classify individual cells in human tonsil tissue into distinct cell clusters using their unique multiplexed protein expression profiles. Furthermore, the analysis disclosed herein revealed that different subregions of the tonsil tissue are composed of cells originating from diverse clusters. Inventors also investigated the associations and avoidance patterns between various cell clusters. Additionally, Inventors observed that the single cells within each cluster can be further partitioned into subclusters based on the distinctive cell clusters of their neighboring cells. These findings highlight the potential of this approach to classify cell types and subtypes by leveraging protein expression profiles and the characteristics of neighboring cells. The identification of these cell types and subtypes holds promise for advancing the understanding of cell heterogeneity, facilitating disease diagnosis, and enabling patient stratification.

The number of imaging cycles and the number of proteins quantified in each cycle are the factors determining the multiplexing capacity of this approach. Inventors have previously reported that the signal erasing treatment and antibody stripping process did not cause any damages to the integrity of the epitopes. Here, Inventors have demonstrated that at least 28 reiterative cycles can be carried out on the same tissue. In each analysis cycle, varied protein targets can be labeled simultaneously with antibodies from varied species or of different immunoglobin classes, hapten or HRP-conjugated primary antibodies. By employing CFTz with four or five distinct fluorophores, along with iterative protein staining, HRP deactivation and fluorophore cleavage, potentially over 10 proteins can be imaged before antibody stripping in every analysis cycle. Consequently, Inventors anticipate that this multiplexed protein imaging technique holds the potential to profile numerous protein targets, potentially in the hundreds, within the same specimen.

The clickable and cleavable fluorescent probes developed here can also be applied for highly sensitive and multiplexed nucleic acid and metabolic imaging. The integration of these technologies and this approach will enable the comprehensive in situ profiling of DNA, RNA, proteins, and metabolites at the single-cell level within intact tissues. Additionally, the incorporation of a program-controlled microfluidic system with a standard fluorescence microscope will make an automated tissue imaging platform. The combination of these advancements forms a highly multiplexed molecular imaging system with broad applications in systems biology and biomedical research.

### Materials and Methods

Chemicals and solvents were obtained from TCI America (Portland, OR, USA) or Sigma-Aldrich (St. Louis, MO, USA) and were directly used without further purification. Bioreagents were purchased from Abcam (Cambridge, United Kingdom), Invitrogen (Waltham, MA, USA), or Novus Biologicals (Littleton, CO, USA), unless otherwise noted.

### Deparaffinization and Antigen Retrieval of FFPE Tonsil Tissue

A total of three xylene deparaffinizations, 10 minutes each, were performed on tonsil FFPE tissue slides (NBP2-30207, Novus Biologicals, Littleton, Colorado, USA) after heating the slide at 60° C. for 1 hr. The slide was subsequently immersed in 50/50 xylene/ethanol, 100% ethanol, 95% ethanol, and 70% ethanol successively, each for 2 minutes, and then rinsed with deionized water. Afterwards, heat-induced antigen retrieval (HIAR) was performed using a microwave. Antigen retrieval citrate buffer (Abcam ab64236) was applied to the slide and heated for 2 minutes and 45 seconds at high power (700 Watt, level 10) and 14 minutes at low power (140 Watt, level 2) then left to room temperature. To deactivate endogenous horse radish peroxidase (HRP) that may cause false positive signals, the slide was incubated with 3% H₂O₂ in PBT (0.1% Triton-X 100 in 1× phosphate buffer saline (PBS)) for 10 minute. Remaining H₂O₂ was washed away with PBT twice before proceeding to protein staining.

### Protein Staining in FFPE Tissue

To avoid non-specific interaction between the surface of the tissue with staining reagents, the slide was treated with antibody blocking buffer (0.1% (vol/vol) Triton X-100, 1% (wt/vol) bovine serum albumin and 10% (vol/vol) normal goat serum) for 30 minutes at room temperature. The corresponding primary antibody to the protein of interest (Table 1) was introduced to the slide at the concentration of 5 μg/mL in antibody blocking buffer for 1 hour. The slide was then washed 3 times in PBT. HRP-Conjugated secondary antibody, either goat anti mouse or goat anti rabbit (Table 1) was incubated with the slide for 30 minutes, followed with three times wash with PBT. Afterwards, the slide were stained with TCO-Tyramide at the concentration of 10 nmol/mL in amplification buffer (0.003% H₂O₂, 0.1% Tween-20, in 100 mM borate, pH=8.5) for 10 min at room temperature, and then washed twice with PBT, each for 5 min. Cleavable fluorophore tetrazine (CFTz) was introduced to the slide at the concentration of 5 nmol/mL amplification buffer (100 mM borate, pH=8.5) for another 10 minutes, and then washed twice with PBT, each for 5 min. To block the uncoupled TCO-Tyramide that might be present in the tissue, 50 nmol/mL of free tetrazine in PBT was added to the tissue and incubated for another 10 minutes, and then washed twice with PBT, each for 5 min. The tissues were stained with DAPI and mounted with Prolong Diamond Antifade Reagent before imaging.

TABLE 1
The antibodies used in this study.

| | Antibody | Catalog | | | |
|---|---|---|---|---|---|
| No. | Target | No. | Host | Conjugation | Source |
| 1 | BRCA1 | 16780 | Mouse | None | Abcam |
| 2 | Ku80 | 232381 | Rabbit | None | Abcam |
| 3 | CD55 | 133684 | Rabbit | None | Abcam |
| 4 | CD79a | 199001 | Mouse | None | Abcam |
| 5 | CD19 | 134114 | Rabbit | None | Abcam |
| 6 | Kat3B | 14984 | Mouse | None | Abcam |
| 7 | CD20 | 279298 | Mouse | None | Abcam |
| 8 | Bcl2 | 182858 | Rabbit | None | Abcam |
| 9 | CD4 | 133616 | Rabbit | None | Abcam |
| 10 | CD45 | 187281 | Mouse | None | Abcam |
| 11 | ILF3 | 206250 | Mouse | HRP | Abcam |
| 12 | NPM1 | 202579 | Mouse | HRP | Abcam |
| 13 | APE1 | 194 | Mouse | None | Abcam |
| 14 | H3S10P | 267372 | Rabbit | None | Abcam |
| 15 | CD11c | 52632 | Rabbit | None | Abcam |
| 16 | H3K9Me2 | 1220 | Mouse | None | Abcam |
| 17 | HLA-DR | 20181 | Mouse | None | Abcam |
| 18 | H3K14Ac | 52946 | Rabbit | None | Abcam |
| 19 | CCR6 | 227036 | Rabbit | None | Abcam |
| 20 | H3K9Ac | 32129 | Rabbit | None | Abcam |
| 21 | H3K18Ac | 40888 | Rabbit | None | Abcam |
| 22 | H3K27Ac | 177178 | Rabbit | None | Abcam |
| 23 | H4K5Ac | 51997 | Rabbit | None | Abcam |
| 24 | H4K8Ac | 45166 | Rabbit | None | Abcam |
| 25 | H4K12Ac | 177793 | Rabbit | None | Abcam |
| 26 | H4K16Ac | 109463 | Rabbit | None | Abcam |
| 27 | Histone H3 | 176842 | Rabbit | None | Abcam |
| 28 | Histone H4 | 177840 | Rabbit | None | Abcam |
| 29 | Rabbit IgG | 6721 | Goat | HRP | Abcam |
| 30 | Mouse IgG | 6789 | Goat | HRP | Abcam |

### Fluorophore Cleavage and HRP Deactivation

100 mM 1,3,5-triaza-7-phosphaadamantane (PTA) and 100 mM tris(2-carboxyethyl) phosphine (TCEP) were successively added to the slide and incubated at 40° C., each for 30 minutes. After incubation, the slide was taken out and washed three times with PBT, each for 5 minutes.

### Antibody Stripping

Antigen retrieval citrate buffer (Abcam ab64236) was added to the slide and heated in the microwave for 2 min and 45 s at high power (level 10, 700 watt) and 14 min at low power (level 2, 140 watt). Then, the slide were submerged in cool water to room temperature for 20 min.

### Synthesis of the Cleavable Fluorescent Tetrazine (CFTz) and Tyramide-TCO (Schemes 1-5)

4-[(1-azido-3-aminopropoxy)methyl]benzoic acid (1): Methyl 4-[(1-azido-3-trifluoroacetamidopropoxy)methyl]benzoate (2) (0.474 g, 1.3 mmol), synthesized according to the literature, was added to a mixture of 4M aqueous sodium hydroxide solution (3.25 mL) and EtOH (3.25 mL). The solution was stirred for 2 hours under room temperature. The solvents were removed by rotary evaporation and the residue was dissolved in 45 mL of D.I. Water. The aqueous layer was extracted with DCM and the organic layer was discarded. The aqueous layer was acidified with 1N HCl to pH 2 and extracted with DCM again. The organic layer was discarded and the aqueous layer was neutralized with 1N NaOH to pH 8. The solution was dried by rotary evaporation and the solids were placed in a funnel with filter paper and washed with 10% MeOH in DCM (45 mL×2) and 50% MeOH in DCM (45 mL×2). The solvents were removed by rotary evaporation to obtain the product in the form of mono-sodium salt as a white solid (0.463 g, yield=123.6%). ¹H-NMR (500 MHZ, MeOD): δ=1.86-1.90 ppm (m, 2H), 2.69-2.71 ppm (t, 2H), 4.58-4.60 ppm (d, 1H), 4.60-4.63 ppm (t, 1H), 4.77-4.79 ppm (d, 1H), 7.31-7.32 ppm (d, 2H), 7.92-7.93 ppm (d, 2H).

N₃-Cy5 (3): Cyanine 5 monoacid (1) (10 mg, 0.013 mmol), DMAP (4 mg, 0.034 mmol) and DSC (5 mg, 0.034 mmol) were dissolved in anhydrous DMF (0.6 mL) and stirred under room temperature for an hour. TLC (CH₃OH:CH₂Cl₂=1:5) was used to check the completion of the reaction. 4-[(1-azido-3-aminopropoxy) methyl]benzoic acid (1) (9.6 mg, 0.034 mmol) and DIPEA (7 μL, 0.041 mmol) was added and the solution was stirred for another hour. The solvent was removed by rotary evaporation and the crude was further purified through preparative silica gel TLC plate (25×25 cm; silica gel 60; CH₃OH:CH₂Cl₂=1:5). Dark blue solid was obtained as the product.

Tetrazine-N₃-Cy5 (5): N₃-Cy5 (3) (11.8 mg, 0.014 mmol), TSTU (6.1 mg, 0.02 mmol) and DIPEA (3.5 μL, 0.02 mmol) was dissolved in anhydrous DMF (0.6 mL) and stirred for an hour at room temperature. TLC (CH₃OH:CH₂Cl₂=1:5) was used to check the completion of the reaction. Methyl tetrazine amine (4 mg, 0.02 mmol) and DIPEA (3.5 μL, 0.02 mmol) was added and the solution was stirred for another hour. The solvent was removed by rotary evaporation and the crude was further purified through preparative preparative silica gel TLC plate (25×25 cm; silica gel 60; CH₃OH:CH₂Cl₂=1:5) to obtain a dark blue solid. The residue was further purified by semi-preparative reverse phase HPLC HPLC gradient: A, 100% 0.1 M TEAA; B 100% MeCN; 0-2 min, 5% B (flow 2-5 ml/min); 2-10 min, 5-22% B (flow 5 ml/min); 10-15 min, 22-30% B (flow 5 ml/min); 15-20 min, 30-40% B (flow 5 ml/min); 20-25 min, 40-50% B (flow 5 ml/min); 25-30 min, 50-60% B (flow 5 ml/min); 30-32 min, 60-70% B (flow 5 ml/min); 32-35 min, 70-95% B (flow 5 ml/min); 35-37 min, 95% B (flow 5 ml/min); 37-39 min, 95-5% B, (flow 5 ml/min); 39-42 min, 5% B (flow 5-2 ml/min)]. The fraction with retention time of 26.40 min was collected and evaporated under reduced pressure to afford Tetrazine-N₃-Cy5 (5) (4.22 mg) as a dark blue solid. ¹H-NMR (500 MHz, MeOD): δ=1.40-1.43 ppm (t, 6H), 1.63-1.66 ppm (t, 2H), 1.77 ppm (s, 12H), 2.11-2.17 ppm (t, 2H), 3.05 ppm (s, 3H), 4.06-4.09 ppm (t, 2H), 4.17-4.21 ppm (q, 2H), 4.68-4.70 ppm (m, 2H), 4.73 ppm (s, 2H), 4.92 ppm (s, 1H), 6.32-6.38 ppm (t, 2H), 6.68-6.73 ppm (t, 1H), 7.31-7.38 ppm (dd, 2H), 7.53-7.55 (d, 2H), 7.65-7.66 (d, 2H), 7.89-7.95 ppm (m, 6H), 8.30-8.35 ppm (t, 2H), 8.53-8.55 ppm (d, 2H). HRMS [+ Scan]; calculated m/z for C₅₄H₆₂N₁₁O₉S₂⁺: 1070.4022; observed m/z: 1070.3948.

N₃—N₃-Cy5 (6): N₃-Cy5 (3) (10.5 mg, 0.012 mmol) (Scheme 5.2), TSTU (6.1 mg, 0.02 mmol) and DIPEA (3.5 μL, 0.02 mmol) was dissolved in anhydrous DMF 0.6 mL and stirred under room temperature for an hour. TLC (CH₃OH:CH₂Cl₂=1:5) was used to check the completion of the reaction. 4-[(1-azido-3-aminopropoxy) methyl]benzoic acid (1) (4.8 mg, 0.017 mmol) and DIPEA (3.5 μL, 0.02 mmol) was added and the solution was stirred for another hour. The solvent was removed by rotary evaporation and the crude was further purified through preparative silica gel TLC plate (25×25 cm; silica gel 60; CH3OH:CH2Cl2=1:5). The product was obtained as a dark blue solid.

Tetrazine-N₃—N₃-Cy5 (7): N₃—N₃-Cy5 (6) (11.1 mg, 0.01 mmol), TSTU (6.1 mg, 0.02 mmol) and DIPEA (3.5 μL, 0.02 mmol) was dissolved in anhydrous DMF 1 mL and stirred under room temperature for an hour. TLC (CH₃OH:CH₂Cl₂=1:5) was used to check the completion of the reaction. Methyl tetrazine amine (2.5 mg, 0.012 mmol) and DIPEA (3.5 μL, 0.02 mmol) was added and the solution was stirred for another hour. The solvent was removed by rotary evaporation and the crude was further purified through preparative TLC (CH₃OH:CH₂Cl₂=1:5). The product was obtained as a dark blue solid. The residue was further purified by semi-preparative reverse phase HPLC HPLC gradient: A, 100% 0.1 M TEAA; B 100% MeCN; 0-2 min, 5% B (flow 2-5 ml/min); 2-10 min, 5-22% B (flow 5 ml/min); 10-15 min, 22-30% B (flow 5 ml/min); 15-20 min, 30-40% B (flow 5 ml/min); 20-25 min, 40-50% B (flow 5 ml/min); 25-30 min, 50-60% B (flow 5 ml/min); 30-32 min, 60-70% B (flow 5 ml/min); 32-35 min, 70-95% B (flow 5 ml/min); 35-37 min, 95% B (flow 5 ml/min); 37-39 min, 95-5% B, (flow 5 ml/min); 39-42 min, 5% B (flow 5-2 ml/min)]. The fraction with retention time of 28.35 min was collected and evaporated under reduced pressure to afford Tetrazine-N₃—N₃-Cy5 (7) (3.21 mg) as a dark blue solid. ¹H-NMR (500 MHz, MeOD): δ=0.86-0.89 ppm (t, 6H), 1.48-1.57 ppm (t, 4H), 1.76 ppm (s, 12H), 2.15-2.196 ppm (t, 4H), 3.06 ppm (s, 3H), 3.36-3.43 ppm (t, 2H), 4.25-4.27 ppm (q, 2H), 4.78-4.80 ppm (t, 2H), 4.84 ppm (s, 1H), 4.94 ppm (s, 1H), 6.33-6.38 ppm (t, 2H), 6.68-6.72 ppm (t, 1H), 7.33-7.38 ppm (dd, 2H), 7.44-7.45 (d, 2H), 7.31-7.53 (d, 2H), 7.66-7.67 ppm (q, 4H), 47.75-7.77 ppm (q, 4H), 8.30-8.37 ppm (t, 2H), 8.55-8.57 ppm (d, 2H). HRMS [+ Scan]; calculated m/z for C₆₅H₇₄N₁₅O₁₁S₂⁺: 1302.4983; observed m/z: 1302.4899.

N₃—N₃—N₃-Cy5 (9): N₃—N₃-Cy5 (6) (16.0 mg, 0.14 mmol), TSTU (9.2 mg, 0.03 mmol) and DIPEA (5.2 μL, 0.03 mmol) was dissolved in anhydrous DMF 0.6 mL and stirred under room temperature for an hour. TLC (CH₃OH:CH₂Cl₂=1:5) was used to check the completion of the reaction. 4-[(1-azido-3-aminopropoxy) methyl]benzoic acid (1) (5.6 mg, 0.02 mmol) and DIPEA (3.5 μL, 0.02 mmol) was added and the solution was stirred for another hour. The solvent was removed by rotary evaporation and the crude was further purified through preparative silica gel TLC plate (25×25 cm; silica gel 60; CH₃OH:CH₂Cl₂=1:5). The product was obtained as a dark blue solid.

Tetrazine-N₃—N₃—N₃-Cy5 (11): N₃—N₃—N₃-Cy5 (9) (13.5 mg, 0.01 mmol), TSTU (6.1 mg, 0.02 mmol) and DIPEA (3.5 μL, 0.02 mmol) was dissolved in anhydrous DMF 1 mL and stirred under room temperature for an hour. TLC (CH₃OH:CH₂Cl₂=1:5) was used to check the completion of the reaction. DIPEA (3.5 μL, 0.02 mmol) and methyl tetrazine amine (2.5 mg, 0.012 mmol) were added and the mixed solution was stirred for another hour. The solvent was removed by rotary evaporation and the crude was further purified through preparative TLC (CH₃OH:CH₂Cl₂=1:5). The product was obtained as a dark blue solid. The residue was further purified by semi-preparative reverse phase HPLC HPLC gradient: A, 100% 0.1 M TEAA; B 100% MeCN; 0-2 min, 5% B (flow 2-5 ml/min); 2-10 min, 5-22% B (flow 5 ml/min); 10-15 min, 22-30% B (flow 5 ml/min); 15-20 min, 30-40% B (flow 5 ml/min); 20-25 min, 40-50% B (flow 5 ml/min); 25-30 min, 50-60% B (flow 5 ml/min); 30-32 min, 60-70% B (flow 5 ml/min); 32-35 min, 70-95% B (flow 5 ml/min); 35-37 min, 95% B (flow 5 ml/min); 37-39 min, 95-5% B, (flow 5 ml/min); 39-42 min, 5% B (flow 5-2 ml/min)]. The fraction with retention time of 33.8 min was collected and evaporated under reduced pressure to afford Tetrazine-N₃—N₃—N₃-Cy5 (11) (1.55 mg) as a dark blue solid. ¹H-NMR (500 MHZ, MeOD): δ=0.86-0.89 ppm (t, 6H), 1.61-1.68 ppm (t, 6H), 1.76 ppm (s, 12H), 2.08-2.19 ppm (m, 6H), 3.06 ppm (s, 3H), 3.46-3.60 ppm (t, 2H), 4.14-4.21 ppm (q, 2H), 4.62-4.68 ppm (t, 1H), 4.70 ppm (s, 2H), 4.72-4.75 ppm (t, 1H), 4.76-4.79 ppm (t, 1H), 4.83 ppm (s, 1H), 4.91-4.92 ppm (s, 1H), 6.35-6.38 ppm (t, 2H), 6.67-6.73 ppm (t, 1H), 7.32-7.36 ppm (dd, 2H), 7.42-7.44 (d, 2H), 7.51-7.53 ppm (d, 2H) 7.63-7.64 ppm (d, 2H), 7.70-7.72 (d, 2H), 7.74-7.75 ppm (d, 2H), 7.89-7.93 ppm (m, 6H), 8.33-8.35 ppm (t, 2H), 8.54-8.55 ppm (d, 2H). HRMS [+ Scan]; calculated m/z for C₇₆H₈₆N₁₉O₁₃S₂⁺: 1534.5943; observed m/z: 1534.6039.

Tyramide-TCO (12): TCO-(PEG)₄-NHS ester (13) (6.0 mg, 0.012 mmol) and Tyramine (14) (2.0 mg, 0.011 mmol) were diluted in 10 μL anhydrous DMF. The mixture was kept in dark room for 2 hours to form the product. The solution was then further diluted in the total of 0.6 mL of DMF as the stock solution for IHC staining.

### Imaging and Data Analysis

A 20×-objective equipped Nikon epifluorescent microscope was wont to image the FFPE tissue. Tissue image was captured by a CooSNAP HQ2 camera and C-FL DAPI HC HISN via Chroma 49009 filter. NIS-Element Imaging Software was used to process the obtained image data. To align all the staining images, DAPI image from each cycle was used as the coordination reference. To generate single cell protein expression profile, cells are defined based on nuclear DAPI staining using NIS Elements Imaging software. Regions of interest (ROIs) were determined by expanding the DAPI signal on every single cell by 10 pixels. Signal intensity values within these ROIs were then calculated via Cell Profiler resulting in a comma separated value (CSV) files. These files were then unsupervisedly clustered to generate Optsne plot (https://doi.org/10.1038/s41467-019-13055-y) pseudo-color images were generated with ImageJ. Cell neighborhoods were calculated by detecting and classifying the encompassing cells within 20 μm or less of every individual cell in the sample. The quantity of cells from the various clusters in each cell neighborhood were used for clustering to come up with subcluster Optsne plots.

### Example 2

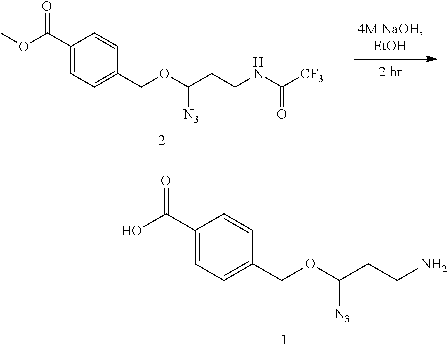 [SMILES: COC(=O)c1ccc(COC(CCNC(=O)C(F)(F)F)N=[N+]=[N-])cc1 ; [N-]=[N+]=NC(CCN)OCc1ccc(C(=O)O)cc1]

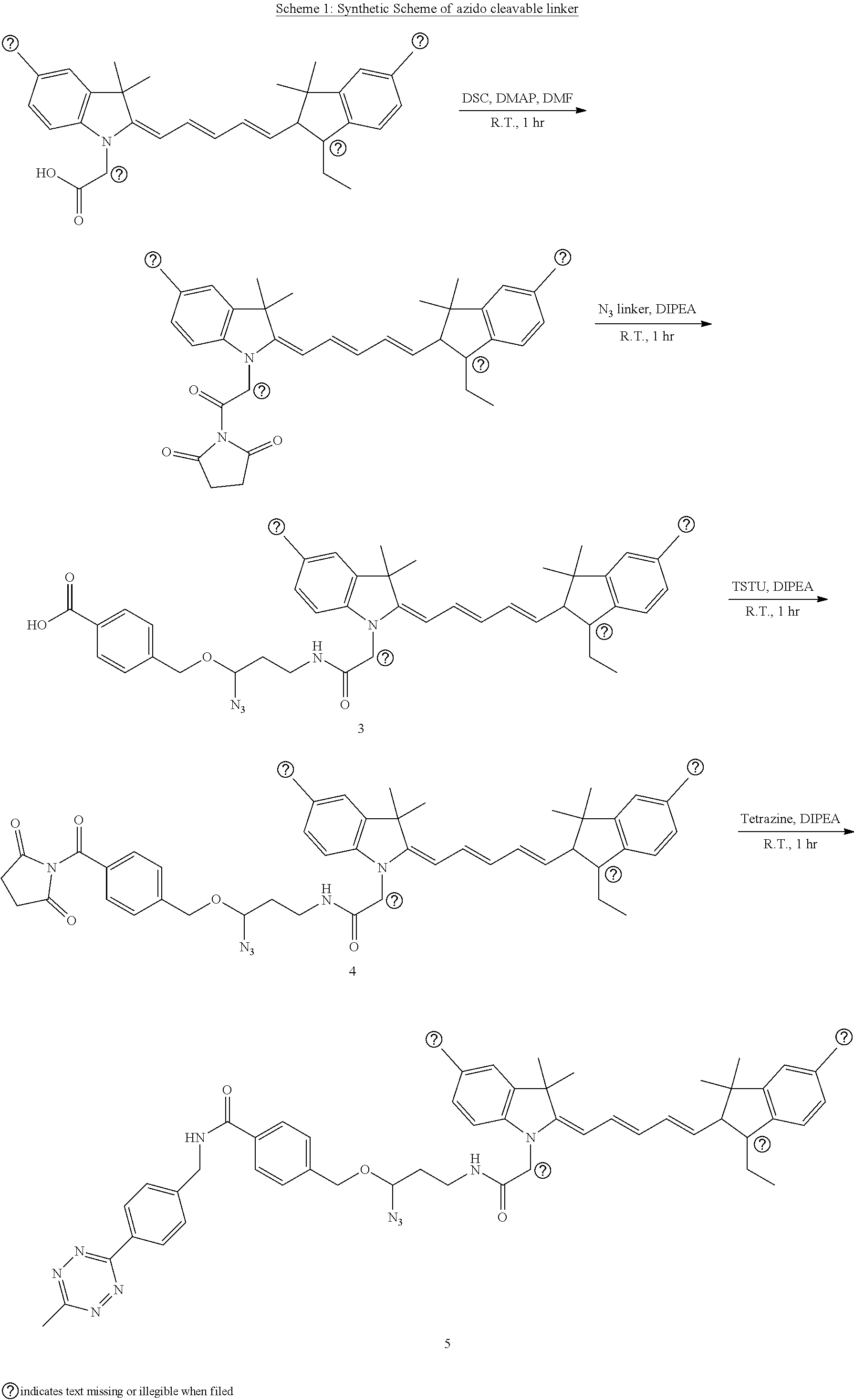

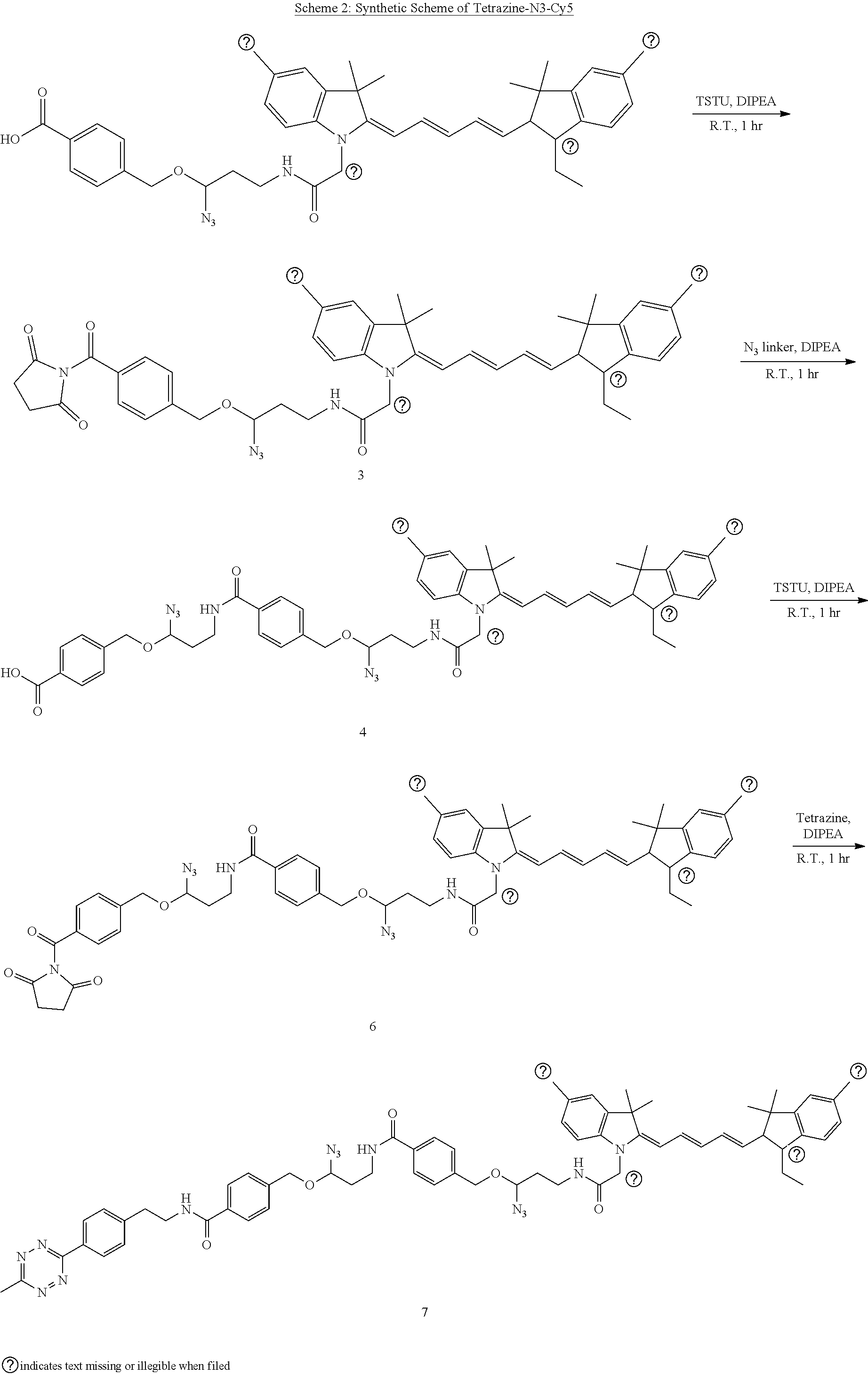

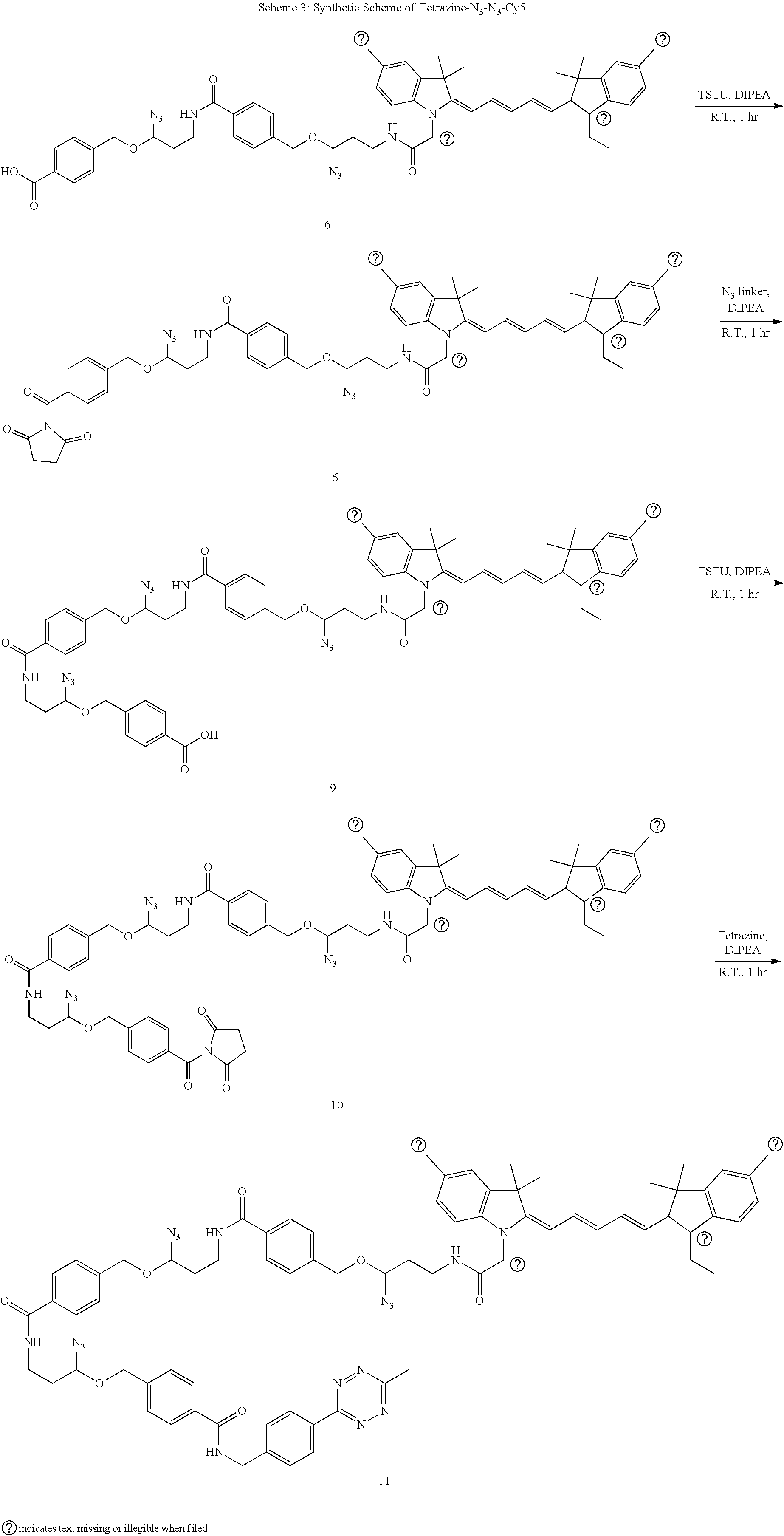

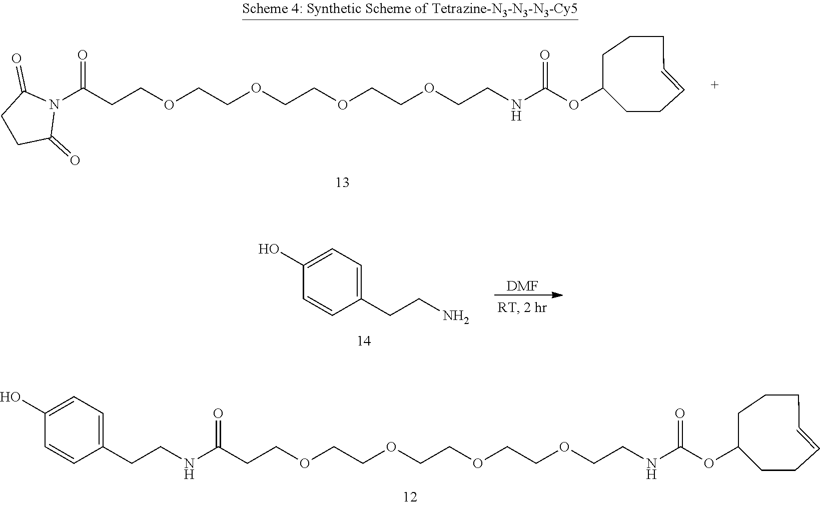

### Scheme 5: Synthetic Scheme of Tyramide-TCO

### Example 3

### Synthetic Procedures

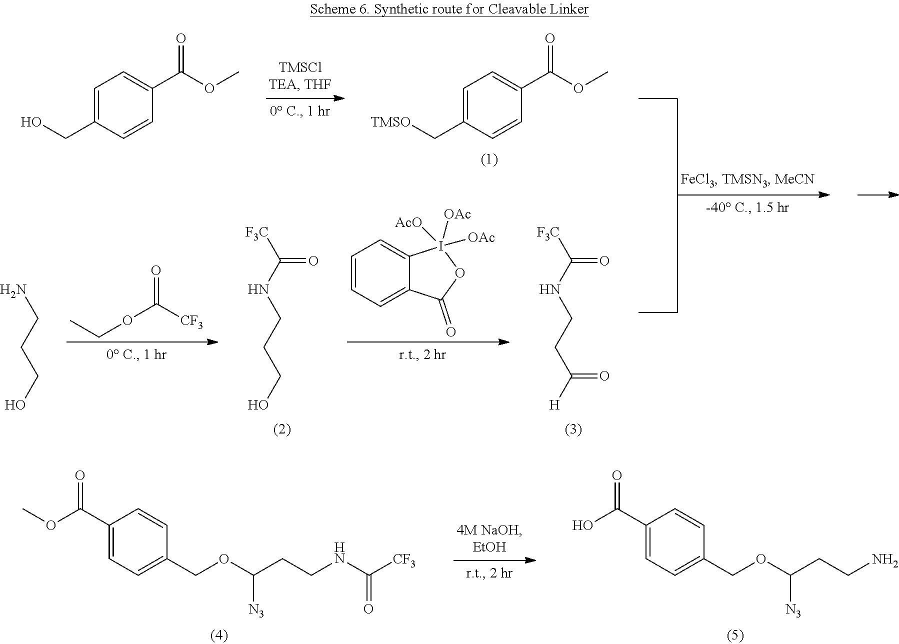

### Methyl 4-[(trimethylsiloxy)methyl]benzoate (1)

Chlorotrimethylsilane (2.3 mL, 18.4 mmol) was added dropwisely to a solution of methyl 4-(hydroxymethyl)benzoate (2.10 g, 12.0 mmol) and triethylamine (3.3 mL, 24.0 mmol) in 80 ml anhydrous THF under ice bath. The mixture was stirred for an hour at 0° C. and 50 mL of hexane was added to the flask. The precipitate was filtered out and the solution was dried by rotary evaporation. The crude was further purified by flash column (DCM:Hex=1:1) to obtain the product as a clear liquid (2.70 g, yield=94.1%). ¹H-NMR (500 MHZ, CDCl₃): δ=0.16 ppm (s, 9H), 3.91 ppm (s, 3H), 4.75 ppm (s, 2H), 7.38-7.39 ppm (d, 2H), 8.00-8.01 ppm (d, 2H).

### N-(3-hydroxypropyl)trifluoroacetamide (2)

Ethyl trifluoroacetate (2.82 g, 20 mmol) was added dropwisely to 3-amino-1-propanol (1.20 g, 16 mmol) and the mixture was stirred at 0° C. for 1 hour. A negative ninhydrin test from the mixture was obtained to determine the completion of the reaction. Volatiles were removed by rotary evaporation and the product was obtained as a clear liquid (2.68 g, yield=98.0%). ¹H-NMR (500 MHZ, CDCl₃): δ=1.80-1.85 ppm (m, 2H), 2.08 ppm (bs, 1H), 3.51-3.56 ppm (q, 2H), 3.80-3.82 ppm (t, 2H).

### N-(3-oxopropyl)trifluoroacetamide (3)

N-(3-hydroxypropyl)trifluoroacetamide (1.6 g, 9.3 mmol) was dissolved in 10 mL anhydrous DCM under ice bath. A solution of Dess-Martin periodinane (45 mL of 10 wt. % in DCM, 14 mmol) was added dropwisely to the solution and stirred for 10 min. The ice bath was removed and the mixture was further stirred for 2 hours under room temperature. The mixture was poured into a biphasic mixture of saturated aqueous Sodium bicarbonate (60 mL) and diethyl ether (60 mL) and stirred for 20 min. The precipitate was filtered out and the layers were separated. The aqueous layer was extracted with diethyl ether and the combined organic layer was washed with brine, dried with magnesium sulfate, and evaporated. The crude was purified by column chromatography (EtOAc:Hex=1:1) to obtain the product as a clear viscous liquid (0.67 g, yield=42.4%). ¹H-NMR (500 MHZ, CDCl₃): δ=2.81-2.83 ppm (t, 2H), 3.61-3.65 ppm (q, 2H), 7.11 ppm (bs, 1H), 9.79 ppm (s, 1H).

### Methyl 4-[(1-azido-3-trifluoroacetamidopropoxy)methyl]benzoate (4)

A solution of trimethylsilyl azide (0.5 mL, 3.75 mmol) and Methyl 4-[(trimethylsiloxy)methyl]benzoate (1) (0.7 g, 2.95 mmol) in anhydrous MeCN 1.5 mL was added dropwisely to a solution of N-(3-oxopropyl)trifluoroacetamide (3) (0.4 g, 2.41 mmol) with catalytic amount of ferric chloride in anhydrous MeCN 5 mL at −40° C. The mixture was stirred for 90 min and quenched with PBS. The aqueous layer was extracted with DCM and the combined organic layer was washed with brine, dried with magnesium sulfate and the volatiles were removed by rotary evaporation. The crude was further purified by column chromatography (DCM:Hex:EtOAc=6:3:1) to obtain the product as a clear liquid (0.474 g, yield=55.6%). ¹H-NMR (500 MHz, CDCl₃): δ=2.06-2.09 ppm (q, 2H), 3.41-3.47 ppm (m, 1H), 3.56-3.60 ppm (m, 1H), 3.90 ppm (s, 3H), 4.61-4.63 ppm (d, 1H), 4.64-4.67 ppm (t, 1H), 7.04 ppm (bs, 1H), 7.39-7.40 ppm (d, 2H), 8.02-8.03 ppm (d, 2H).

### 4-[(1-azido-3-aminopropoxy)methyl]benzoic acid (5)

Methyl 4-[(1-azido-3-trifluoroacetamidopropoxy)methyl]benzoate (4) (0.474 g, 1.3 mmol) was added to a mixture of 4M aqueous sodium hydroxide solution 3.25 mL and EtOH 3.25 mL. The solution was stirred for 2 hours under room temperature. The solvents were removed by rotary evaporation and the residue was dissolved in D.I. Water 45 mL. The aqueous layer was extracted with DCM and the organic layer was discarded. The aqueous layer was acidified with 1N HCl to pH 2 and extracted with DCM again. The organic layer was discarded and the aqueous layer was neutralized with 1N NaOH to pH 8. The solution was dried by rotary evaporation and the solids were placed in a funnel with filter paper and washed with 10% MeOH in DCM (45 mL×2) and 50% MeOH in DCM (45 mL×2). The solvents were removed by rotary evaporation to obtain the product in the form of mono-sodium salt as a white solid (0.463 g, yield=123.6%). ¹H-NMR (500 MHZ, MeOD): δ=1.86-1.90 ppm (m, 2H), 2.69-2.71 ppm (t, 2H), 4.58-4.60 ppm (d, 1H), 4.60-4.63 ppm (t, 1H), 4.77-4.79 ppm (d, 1H), 7.31-7.32 ppm (d, 2H), 7.92-7.93 ppm (d, 2H).

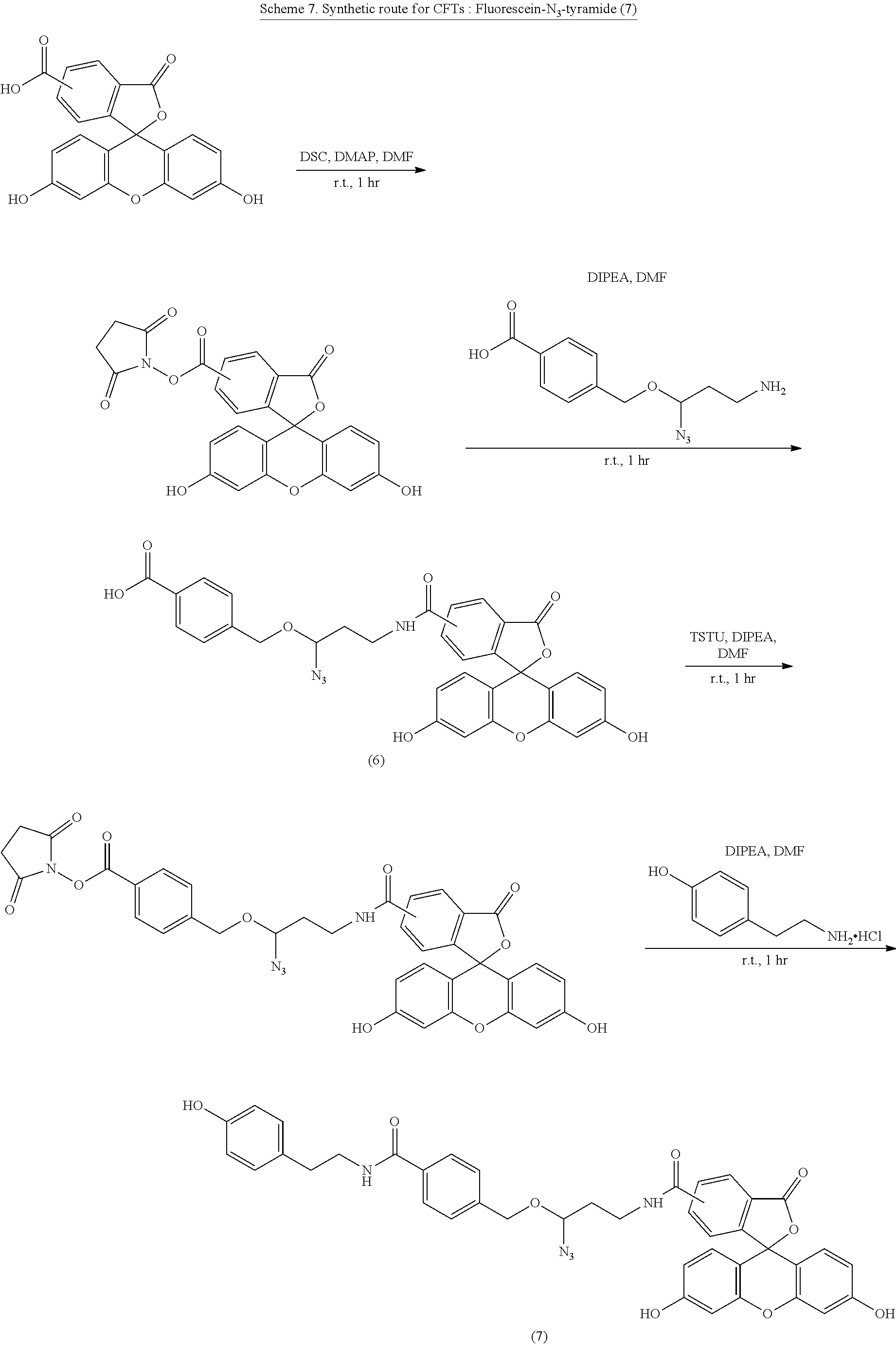

### Fluorescein-N₃ (6)

5(6)-Carboxyfluorescein (10 mg, 0.027 mmol), DSC (9 mg, 0.034 mmol) and DMAP (4 mg, 0.034 mmol) were dissolved in anhydrous DMF 1 mL and stirred under room temperature for an hour. TLC (DCM:MeOH=3:1) was used to check the completion of the first step reaction. 4-[(1-azido-3-aminopropoxy)methyl]benzoic acid (5) (9.6 mg, 0.034 mmol) and DIPEA (7 μL, 0.041 mmol) was added and the solution was stirred for another hour. The solvent was removed by rotary evaporation and the crude was further purified through preparative TLC (DCM:MeOH=3:1). The product was obtained as a yellow solid.

### Fluorescein-N₃-Tyramide (7)

Fluorescein-N₃ (6) (16.4 mg, 0.027 mmol), TSTU (12.2 mg, 0.041 mmol) and DIPEA (7 μL, 0.041 mmol) was dissolved in anhydrous DMF 1 mL and stirred under room temperature for an hour. TLC (DCM:MeOH=5:1) was used to check the completion of the first step reaction. Tyramine hydrochloride (9.4 mg, 0.054 mmol) and DIPEA (9 μL, 0.054 mmol) was added and the solution was stirred for another hour. The solvent was removed by rotary evaporation and the crude was further purified through preparative TLC (DCM:MeOH=5:1) and HPLC. The product was obtained as a yellow solid. HRMS [+ Scan]; calculated m/z for C₄₀H₃₃N₅O₉: 728.2351; observed m/z: 728.2291.

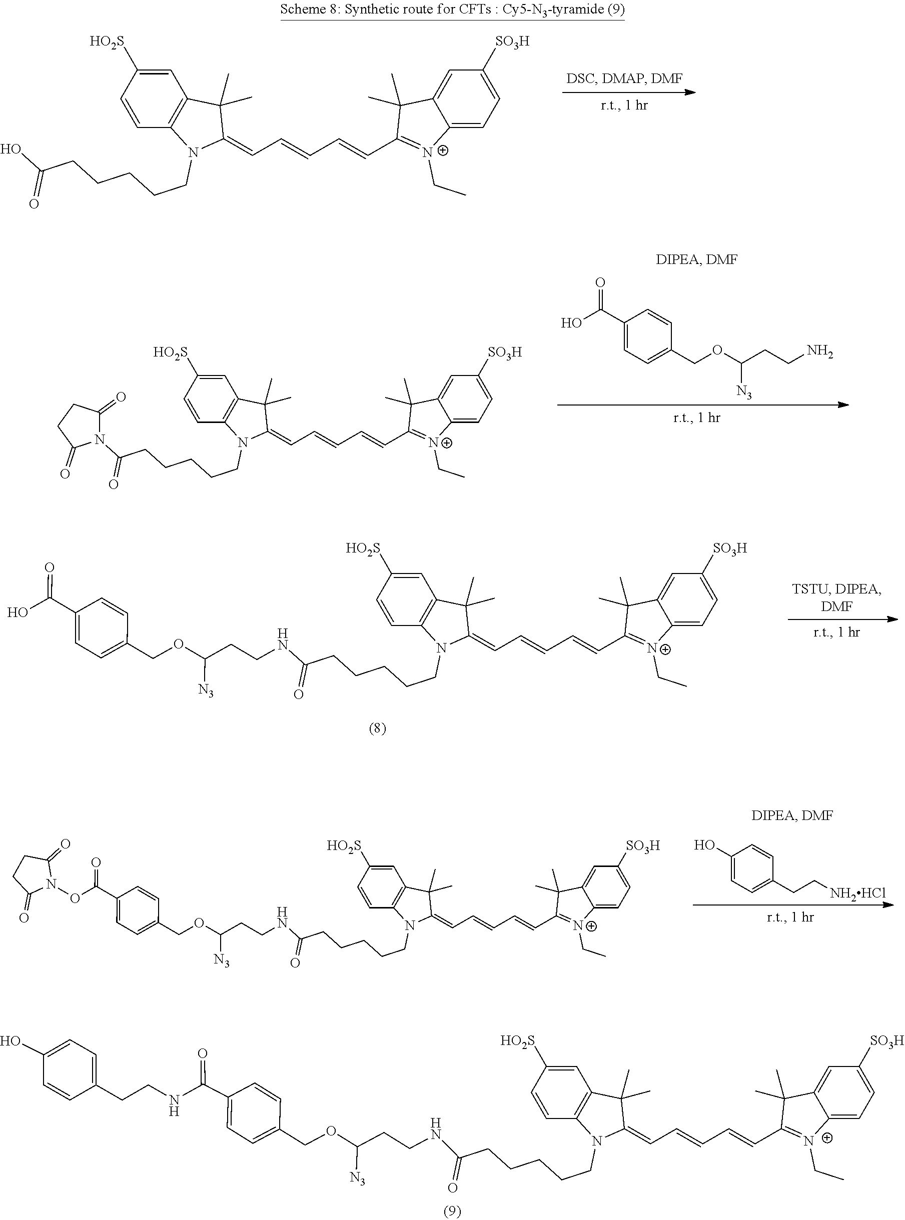

### Cy5-N₃ (8)

Sulfo-Cyanine5 (10 mg, 0.013 mmol), DSC (5 mg, 0.019 mmol) and DMAP (2 mg, 0.019 mmol) were dissolved in anhydrous DMF 1 mL and stirred under room temperature for an hour. TLC (DCM:MeOH=3:1) was used to check the completion of the first step reaction. 4-[(1-azido-3-aminopropoxy)methyl]benzoic acid (5) (5.4 mg, 0.019 mmol) and DIPEA (4 μL, 0.023 mmol) was added and the solution was stirred for another hour. The solvent was removed by rotary evaporation and the crude was further purified through preparative TLC (DCM:MeOH=3:1). The product was obtained as a blue solid.

### Cy5-N₃-Tyramide (9)

Cy5-N₃ (6) (11.5 mg, 0.013 mmol), TSTU (6.8 mg, 0.023 mmol) and DIPEA (4 μL, 0.023 mmol) was dissolved in anhydrous DMF 1 mL and stirred under room temperature for an hour. TLC (DCM:MeOH=5:1) was used to check the completion of the first step reaction. Tyramine hydrochloride (4.5 mg, 0.026 mmol) and DIPEA (4.3 μL, 0.026 mmol) was added and the solution was stirred for another hour. The solvent was removed by rotary evaporation and the crude was further purified through preparative TLC (DCM:MeOH=5:1) and HPLC. The product was obtained as a pink solid. HRMS [+ Scan]; calculated m/z for C₅₂H₆₂N₇O₁₀S₂: 1008.4000; observed m/z: 1008.4039.

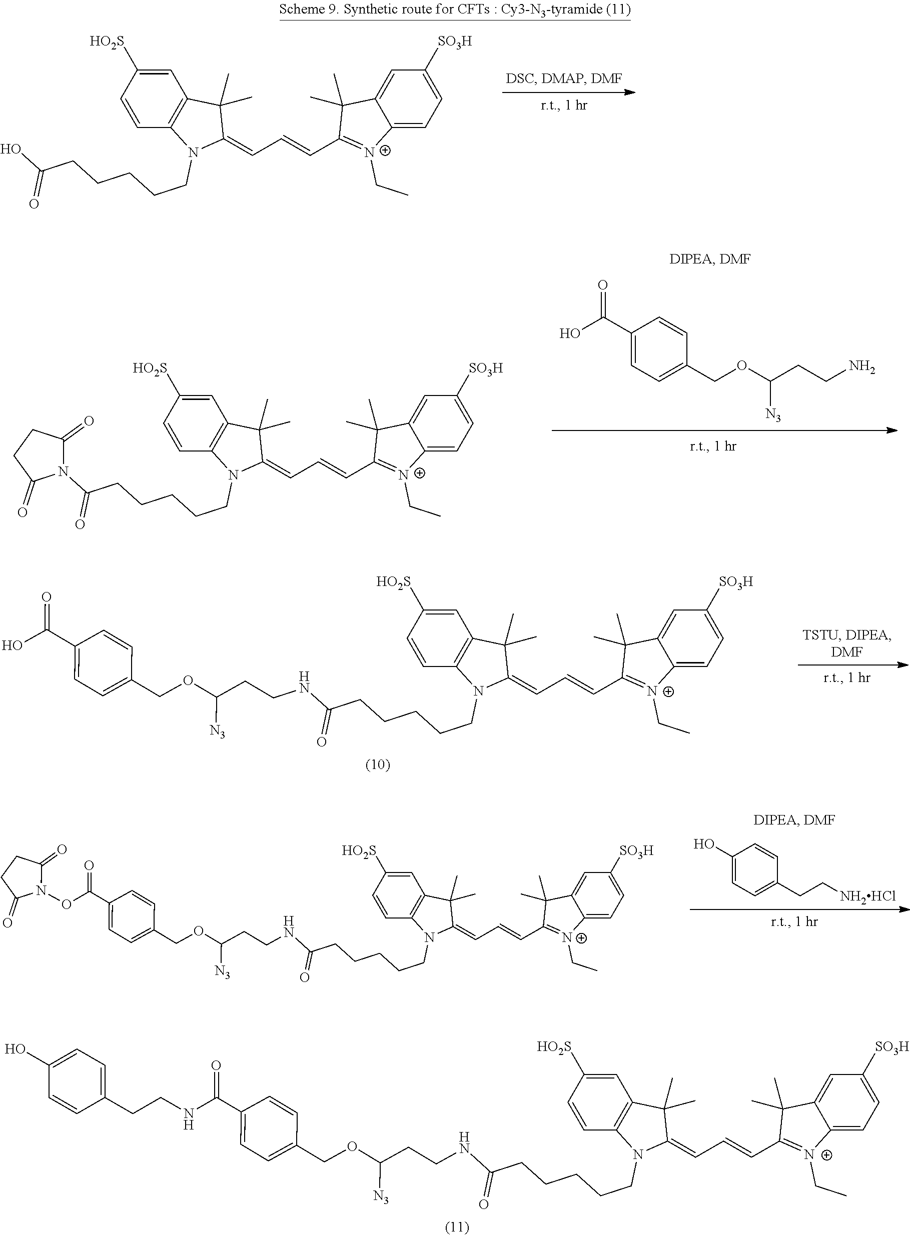

### Cy3-N₃ (10)

Sulfo-Cyanine3 (10 mg, 0.013 mmol), DSC (5 mg, 0.019 mmol) and DMAP (2 mg, 0.019 mmol) were dissolved in anhydrous DMF 1 mL and stirred under room temperature for an hour. TLC (DCM:MeOH=3:1) was used to check the completion of the first step reaction. 4-[(1-azido-3-aminopropoxy)methyl]benzoic acid (5) (5.4 mg, 0.019 mmol) and DIPEA (4 μL, 0.023 mmol) was added and the solution was stirred for another hour. The solvent was removed by rotary evaporation and the crude was further purified through preparative TLC (DCM:MeOH=3:1). The product was obtained as a pink solid.

### Cy3-N₃-Tyramide (11)

Cy3-N₃ (6) (11.1 mg, 0.013 mmol), TSTU (6.8 mg, 0.023 mmol) and DIPEA (4 μL, 0.023 mmol) was dissolved in anhydrous DMF 1 mL and stirred under room temperature for an hour. TLC (DCM:MeOH=5:1) was used to check the completion of the first step reaction. Tyramine hydrochloride (4.5 mg, 0.026 mmol) and DIPEA (4.3 μL, 0.026 mmol) was added and the solution was stirred for another hour. The solvent was removed by rotary evaporation and the crude was further purified through preparative TLC (DCM:MeOH=5:1) and HPLC. The product was obtained as a pink solid. HRMS [+ Scan]; calculated m/z for C₅₀H₆₀N₇O₁₀S₂: 982.3838.

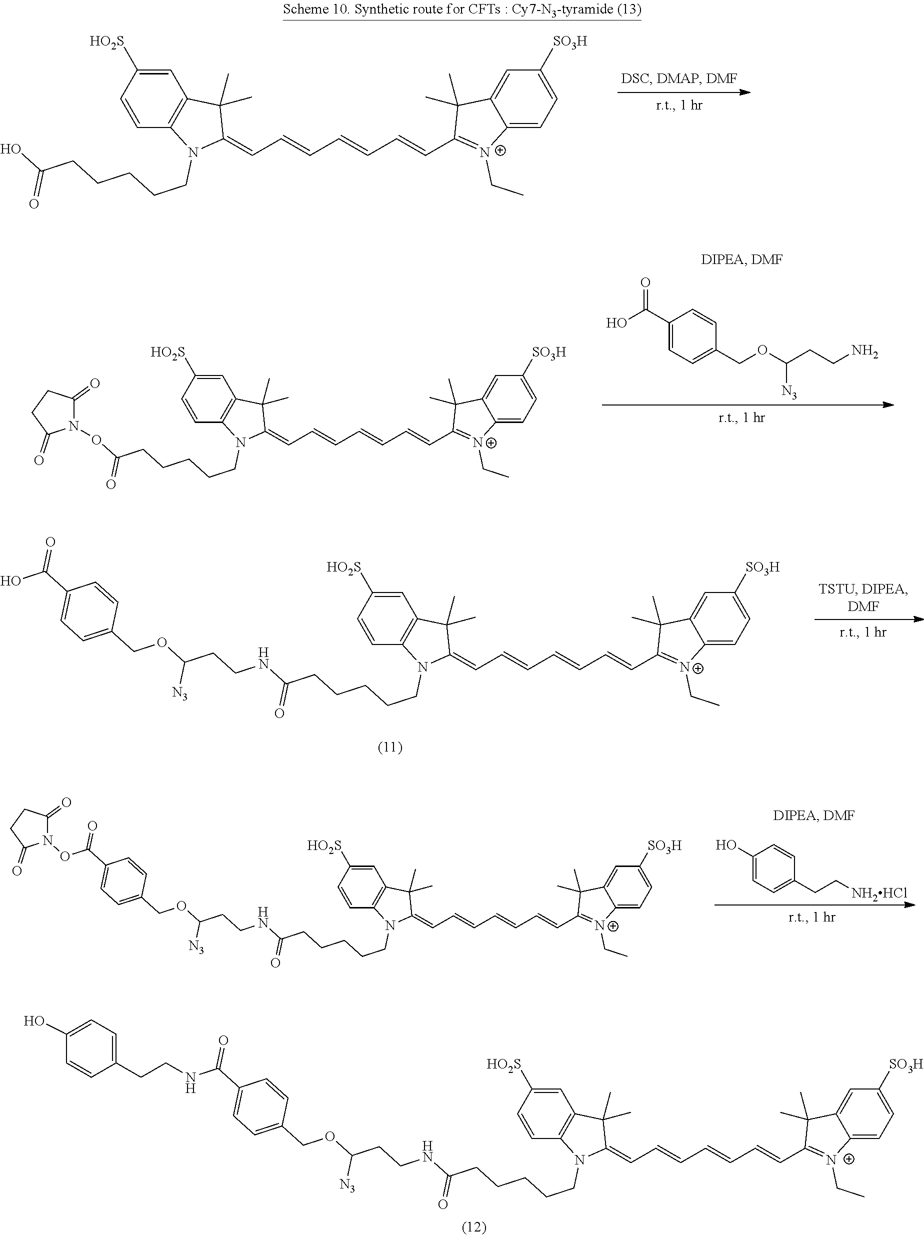

### Cy7-N₃ (12)

Sulfo-Cyanine7 (10 mg, 0.013 mmol), DSC (5 mg, 0.019 mmol) and DMAP (2 mg, 0.019 mmol) were dissolved in anhydrous DMF 1 mL and stirred under room temperature for an hour. TLC (DCM:MeOH=3:1) was used to check the completion of the first step reaction. 4-[(1-azido-3-aminopropoxy)methyl]benzoic acid (5) (5.4 mg, 0.019 mmol) and DIPEA (4 μL, 0.023 mmol) was added and the solution was stirred for another hour. The solvent was removed by rotary evaporation and the crude was further purified through preparative TLC (DCM:MeOH=3:1). The product was obtained as a green solid.

### Cy7-N₃-Tyramide (13)

Cy7-N₃ (6) (11.1 mg, 0.013 mmol), TSTU (6.8 mg, 0.023 mmol) and DIPEA (4 μL, 0.023 mmol) was dissolved in anhydrous DMF 1 mL and stirred under room temperature for an hour. TLC (DCM:MeOH=5:1) was used to check the completion of the first step reaction. Tyramine hydrochloride (4.5 mg, 0.026 mmol) and DIPEA (4.3 μL, 0.026 mmol) was added and the solution was stirred for another hour. The solvent was removed by rotary evaporation and the crude was further purified through preparative TLC (DCM:MeOH=5:1) and HPLC. The product was obtained as a green solid. HRMS [+ Scan]; calculated m/z for C₅₀H₆₀N₇O₁₀S₂: 982.3838.

We claim:

1. A system, kit, platform, or composition comprising:
a first component comprising a tyramide conjugated to a trans-cylooctene; and
a second component,
wherein the second component has Formula (II): 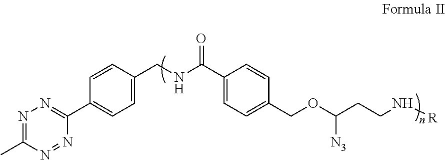
wherein R comprises a detectable marker; and
wherein n is 1 to 3.

2. The system, kit, platform, or composition of claim 1, wherein the first component has Formula (I):

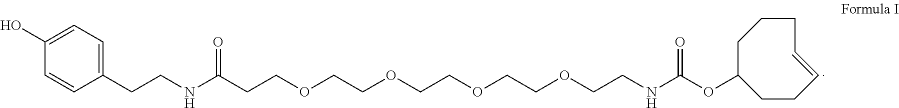 [SMILES: O=C(CCOCCOCCOCCOCCNC(=O)OC1CC/C=C/CCC1)NCCc1ccc(O)cc1]

3. (canceled)

4. The system, kit, platform, or composition of claim 1, wherein the detectable marker is selected from the group consisting of Cy5, TAMRA (labeled with tetramethylrhodamine or “TMR”), ALEXA FLUOR™ 594, and ATTO 647N and ATTO 700 fluorophores (ATTO-TEC, Germany), quantum dots, ALEXA FLUOR™ 350, ALEXA FLUOR™ 532, ALEXA FLUO^{R™} 546, ALEXA FLUOR™ 568, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, DYLIGHT™ DYES (e.g., DYLIGHT™ 405, DYLIGHT™ 488, DYLIGHT™ 549, DYLIGHT™ 594, DYLIGHT™ 633, DYLIGHT™ 649, DYLIGHT™ 680, DYLIGHT™ 750, DYLIGHT™ 800 and the like), Texas Red, and Cy2, Cy3.5, Cy5.5, Cy7, sulfonated Cy2, Cy3.5, Cy 5, Cy5.5, and Cy7, fluorescent proteins, and radioisotopes.

5. The system, kit, platform, or composition of claim 1, wherein the detectable marker comprises a fluorophore.

6. The system, kit, platform, or composition of claim 5, wherein the fluorophore is selected from the group consisting of Cy5, TAMRA, ALEXA FLUOR™ 594, ATTO 647N, and ATTO 700.

7. The system, kit, platform, or composition of claim 5, wherein the second component has Formula (III): 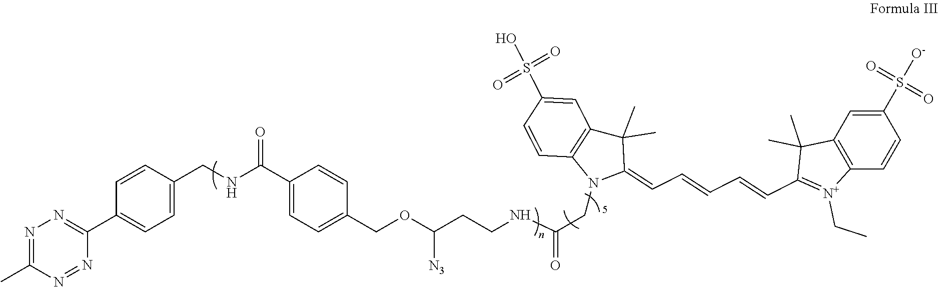
wherein n is 1 to 3.

8. A method of multiplex in situ analysis of biomolecules in a tissue sample, the method comprising:
(a) contacting a tissue sample with a first plurality of horseradish peroxidase (HRP)-conjugated targeting agents that are configured to specifically bind to or hybridize to a first target biomolecule in the tissue sample under conditions that promote binding or hybridization of the targeting agents to the target biomolecule;
(b) contacting the tissue sample with the compound of Formula I under conditions that promote conjugation of the compound to the target biomolecule;
(c) contacting the tissue sample with the compound of claim 1 under conditions that promote conjugation of the compound of claim 1 to the compound of Formula I;
(d) imaging the tissue sample thereby detecting the detectable marker;
(e) contacting the tissue sample with a composition comprising 1,3,5-Triaza-7-phosphaadamantane (PTA) and tris(2-carboxyethyl) phosphine (TCEP);
(f) repeating steps (a)-(e); wherein a second plurality of HRP-conjugated targeting agents is used to bind to or hybridize to a second target biomolecule, wherein the first and the second target biomolecules are different.

9. The method of claim 8, further comprising:
after step (e) and before step (f), contacting the tissue sample with a plurality of unlabeled tetrazine thereby blocking unbound compound of Formula I.

10. The method of claim 8, further comprising:
(g) repeating steps (a)-(e) N times, wherein the Nth plurality of HRP-conjugated targeting agents is used to bind to or hybridize to the Nth target biomolecule, wherein the first through the Nth target biomolecules are different.

11. The method of claim 8, wherein the first plurality of targeting agents comprises HRP-conjugated synthetic DNA oligonucleotide probes.

12. The method of claim 8, wherein the first plurality of targeting agents comprises HRP-conjugated polyclonal antibodies, HRP-conjugated monoclonal antibodies, or HRP-conjugated antigen-binding fragments thereof.

13. The method of claim 8, wherein the first target biomolecules are less abundant in the sample tissue than the second target biomolecules.

14. The method of claim 8, wherein step (e) comprises incubating the contacted sample at about 40° C. for about 30 minutes.

15. A kit comprising:
(a) a composition comprising the compound of Formula I and Formula II;
(b) a composition comprising 1,3,5-Triaza-7-phosphaadamantane (PTA); and
(c) a composition comprising tris(2-carboxyethyl) phosphine (TCEP).

16. The kit of claim 15, further comprising:
(d) horseradish peroxidase (HRP)-conjugated targeting agents.

17. (canceled)

18. A compound having Formula (II): 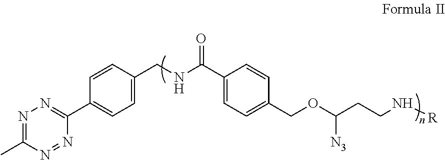
wherein R comprises a detectable marker; and
wherein n is 1 to 3.

19. (canceled)

20. The compound of claim 18, wherein the detectable marker comprises a fluorophore.

21. (canceled)

22. A compound having Formula (III): 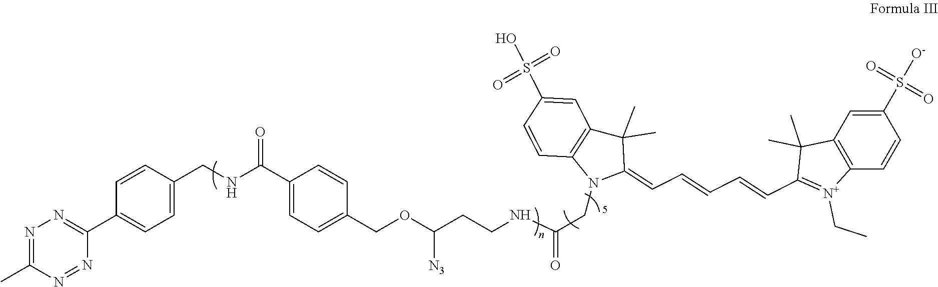
wherein n is 1 to 3.
